SEQUENCE LISTING (20 sequences)

SEQ ID NO: 1 - DNA, 24 bp, Artificial sequence
  Single strand DNA oligonucleotide primer
ggatccttccaggcacagtgtcac

SEQ ID NO: 2 - DNA, 26 bp, Artificial sequence
  Single strand DNA oligonucleotide primer
ggtacccacatgcctgctcccgcgtg

SEQ ID NO: 3 - DNA, 27 bp, Artificial sequence
  Single strand DNA oligonucleotide primer
gcggccgcacgcccgcctgtccgcagg

SEQ ID NO: 4 - DNA, 24 bp, Artificial sequence
  Single strand DNA oligonucleotide primer
gtaggaggaataggcgatgcgcgc

SEQ ID NO: 5 - DNA, 27 bp, Artificial sequence
  Single strand DNA oligonucleotide primer
ggagctcttctacagcgtcaccatggc

SEQ ID NO: 6 - DNA, 21 bp, Artificial sequence
  Single strand DNA oligonucleotide primer
cgcaccgcccccggtcgtcgc

SEQ ID NO: 7 - DNA, 24 bp, Artificial sequence
  Single strand DNA oligonucleotide primer
ggtcctcgccctcttcctgaagcg

SEQ ID NO: 8 - DNA, 25 bp, Artificial sequence
  Single strand DNA oligonucleotide primer
cgtagtagaccaccaggtagtagcc

SEQ ID NO: 9 - DNA, 23 bp, Artificial sequence
  Single strand DNA oligonucleotide primer
gcgcatgaatcctggcccaggcg

SEQ ID NO: 10 - DNA, 25 bp, Artificial sequence
  Single strand DNA oligonucleotide primer
cgagggaatggcgtacccagcagcg

SEQ ID NO: 11 - DNA, 24 bp, Artificial sequence
  Single strand DNA oligonucleotide primer
tctcaggcgccatcacgtccttcg

SEQ ID NO: 12 - DNA, 24 bp, Artificial sequence
  Single strand DNA oligonucleotide primer
actcacgtggcgatgggcacgagg

SEQ ID NO: 13 - DNA, 24 bp, Artificial sequence
  Single strand DNA oligonucleotide primer
ttgccgctttcagattgcatcttc

SEQ ID NO: 14 - DNA, 22 bp, Artificial sequence
  Single strand DNA oligonucleotide primer
tgaacccttgcggaccgggagg

SEQ ID NO: 15 - DNA, 23 bp, Artificial sequence
  Single strand DNA oligonucleotide primer
cctcacccggcttctctttccag

SEQ ID NO: 16 - DNA, 24 bp, Artificial sequence
  Single strand DNA oligonucleotide primer
ggaaagcggcgggctctgggctgg

SEQ ID NO: 17 - DNA, 23 bp, Artificial sequence
  Single strand DNA oligonucleotide primer
ggacaagggcctcggaggcctgc

SEQ ID NO: 18 - DNA, 23 bp, Artificial sequence
  Single strand DNA oligonucleotide primer
ggacagccagctggggacaagtc

SEQ ID NO: 19 - DNA, 23 bp, Artificial sequence
  Single strand DNA oligonucleotide primer
gcagagacagagcgacccatacc

SEQ ID NO: 20 - DNA, 22 bp, Artificial sequence
  Single strand DNA oligonucleotide primer
gcaaagttaccacaggggcgcc

(19) United States
(12) Patent Application Publication
(10) Pub. No.: US 2004/0101834 A1
(43) Pub. Date: May 27, 2004

(54) Method of and kit for assessing responsiveness of cancer patients to antifolate chemotherapy

(76) Inventors: Yehuda Assaraf, Doar Na Misgav (IL); Stavit Drori, Haifa (IL)

Correspondence Address: Anthony Castorina, G E Ehrlich, Suite 207, 2001 Jefferson Davis Highway, Arlington, VA, 22202, US

(21) Appl. No.: 09/936,764

(22) PCT Filed: Mar. 6, 2001
(86) PCT No.: PCT/IL01/00212

(51) Int. Cl. C12Q 1/68

(52) U.S. Cl. 435/6

(57) ABSTRACT

A method for the assessment of the responsiveness of a cancer patient to antifolate-containing chemotherapy, the method is effected by searching for a mutation or mutations in a reduced folate carrier (RFC) gene in cells derived from the patient.

### FIELD AND BACKGROUND OF THE INVENTION

The present invention relates to a method of and a kit for assessing the responsiveness of cancer patients to antifolate chemotherapy. More particularly, the present invention relates to the detection of mutations in genes associated with folate and folate analog (e.g., antifolate) uptake or metabolism, such as the reduced folate carrier (RFC) gene, of patients or of cancer cell samples or biopsies thereof.

Folates are essential co-factors in eukaryotic cells, serving as one-carbon donors in several biosynthetic processes, including purine and pyrimidine biosynthesis, mitochondrial protein synthesis and amino acid conversion (Stokstad, 1990). Biologically active folates predominantly exist in a reduced (tetrahydrofolate) form and are retained intracellularly in a polyglutamate form. The finding that folates are essential vitamins for growth and proliferation of neoplastic cells has been exploited for the design and clinical application of folate analogs as chemotherapeutic drugs (Jansen, 1999). Since the first remissions from childhood acute lymphoblastic leukemias (ALL) that were induced by aminopterin, folic acid antagonists in general, and methotrexate (MTX) in particular (FIG. 1), have become increasingly important in cancer chemotherapy (Jansen, 1999). MTX has an established role in the treatment of various malignancies including childhood ALL, non-Hodgkin's lymphoma, osteogenic sarcoma, head and neck cancer, choriocarcinoma, small cell lung cancer, and breast cancer (Bertino, 1993). MTX is also applied in some non-malignant disorders, such as psoriasis, rheumatoid arthritis and non-steroid dependent asthma (Banwarth et al, 1994). Furthermore, MTX treatment is not associated with severe long-term toxicity, which is an important factor in curative chemotherapy and in adjuvant chemotherapy applied for treatment of breast cancer (Bertino, 1993; Schornagel and McVie, 1983).

As is evident from FIG. 1, folates (e.g. folic acid) and antifolates (e.g., methotrexate) are of divalent anionic nature and thus require specific transport proteins for their translocation across biological membranes. Several transport systems have been described in various mammalian model cell lines that can accommodate transport of folates and their folate-based chemotherapeutic agents, including MTX (Goldman and Matherly, 1985; Sirotnak, 1985; Jansen, 1999).

The reduced folate carrier (RFC) serves as the major uptake route for folate cofactors and antifolate anticancer drugs. RFC functions as a bidirectional anion exchanger (Goldman, 1971; Henderson and Zevely, 1981) and is described hereinbelow in greater detail.

Folate receptors, glycosylphosphatidylinositol membrane-anchored proteins that mediate the unidirectional uptake of folates display high affinity for folic acid and 5-methyltetrahydrofolate (K_{D}=1−10 nM), but lower affinity (K_{D}=10−300 nM) for other reduced folates and MTX (Antony, 1992; Brigle et al., 1994; Wang et al., 1992; Westerhof et al., 1995).

In addition, there is an apparently independent folate transport system with optimal uptake activity at low pH which recognizes folic acid, reduced folates, and MTX with comparable affinities (Kₘ=1−5 μM) (Henderson and Strauss, 1990; Sierra et al., 1997; Kumar et al., 1997; Assaraf et al., 1998).

Intracellular (anti)folate Metabolism:

Major pathways of folate metabolism in the cytosol and in mitochondria of eukaryotes are summarized in FIG. 2, which includes the following:

Target enzymes for antifolates: Dihydrofolate reductase (DHFR) is the main intracellular target for MTX. MTX binds (stoichiometrically) to the enzyme with a 10⁶-fold higher affinity than the natural substrate dihydrofolate (Ozaki, 1993). Studies have shown that at least 95% of the enzyme should be inhibited in order to deplete intracellular tetrahydrofolate pools and thereby block cell growth (Jackson and Harrap, 1973). In the past two decades other enzymes in folate metabolism have been recognized as targets for antifolates, e.g. thymidylate synthase (TS), glycinamide ribonucleotide transformylase (GARTF) and folylpoly γ-glutamate synthetase (FPGS).

Polyglutamylation: Intracellular folates and antifolate anticancer drugs exist mainly as poly-γ-glutamate derivatives. The (anti)folate molecule is elongated by up to 10 glutamyl residues connected therebetween by peptide bonds through the activity of FPGS, as follows:

Folate(glu)ₙ+MgATP+L-glutamic acid→Folate(glu)ₙ₊₁+MgADP+Pi

The conjugation of multiple glutamate residues renders the resulting (anti)folate a polyanionic compound which is therefore retained intracellularly (Chabner et al., 1985; Fry et al., 1983; Poser et al., 1981).

Polyglutamylation of natural reduced folate cofactors is crucial for cell growth. This is illustrated by studies in which Chinese hamster ovary (CHO) cells transfected with Escherichia coli FPGS (which is capable of polyglutamylation only up to three Glu residues) appeared to be auxotrophic for glycine and methionine. Other transfectants with higher FPGS levels and higher intracellular concentrations of long chain polyglutamates could grow in the absence of methionine (Lowe et al., 1993).

In general, the binding affinity of folate-dependent enzymes for folate and antifolate compounds increases with increasing polyglutamate chain length (Chabner et al., 1985; Fry et al., 1983; Shane 1989). MTX-polyglutamates per se are no better inhibitors of DHFR than the monoglutamate form. However, they are more potent inhibitors of other folate dependent enzymes such as TS (Allegra, 1985a) and phosphoribosylamino-imidazole-carboxamide transformylase (AICAR) (Allegra, 1985b; Allegra, 1987; Baggott et al., 1986). The TS inhibitor, ZD1694 (Tomudex, Raltitrexed), is also a more potent inhibitor when polyglutamated: ZD1694-Glu5 has a Ki of 1 nM, whereas the parent compound has a Ki of 60 nM (Jackman, 1991). It has also been shown that high levels of dihydrofolate polyglutamates formed after DHFR inhibition can be potent inhibitors of TS and AICAR )Allegra 1985a).

In general, FPGS activity is higher in proliferating, embryonic and tumor cells as compared to non-proliferating and quiescent cells (Poser et al., 1981; Fry et al., 1983; Matherly et al., 1986; Matherly et al., 1987; Egan et al., 1995; Fabre et al., 1984; Barredo et al., 1994; Jansen et al., 1992). Along with the increased potency of polyglutamated antifolates, tumor cells with increased FPGS levels are more sensitive to folate-based chemotherapy. Uptake and polyglutamylation of MTX in children with ALL have been recognized as prognostic factors for treatment outcome (Whitehead et al., 1990; Whitehead et al., 1992; Li et al., 1992; Lin and Bertino, 1991). Thus, studies by Whitehead et al., demonstrated that children with ALL displaying higher MTX uptake and MTX polyglutamate formation, had a better five-year survival rate than children who scored lower for these parameters (Whitehead et al., 1990; Whitehead et al., 1991). In addition, in B-lineage ALL, accumulation of MTX-pentaglutamates is doubled in hyperdiploid cells as is compared to non-hyperdiploid cells (Synold et al., 1994). FPGS levels, measured by RT-PCR-analysis, in blast cells from 11 patients with ALL and acute myelogenous leukemia, showed a wide expression range of about 500-fold (Lenz et al., 1994). The large range of FPGS expression in clinical samples could have clinical implications; it could predict to what extent patients may respond to antifolate-containing chemotherapy (Rots et al., 1999a, b).

Mechanisms of Resistance:

As with many other anticancer drugs, the successful treatment of neoplastic diseases by antifolate-containing chemotherapy is often limited by the frequent emergence of drug resistance. Resistance can be divided into two categories: (i) intrinsic (or pre-existing), where tumor cells do not respond to the drug, not even during the first exposure to the drug; and (ii) acquired, in which tumor cells initially respond well to the drug but become insensitive thereto during or after the chemotherapeutic treatment.

The mechanisms of resistance to antifolates in general, and to MTX in particular, can be divided into four main groups: (i) impaired drug uptake; (ii) overproduction of a target-enzyme; (iii) structurally altered target enzyme; and (iv) decreased polyglutamylation (Goldman and Matherly, 1985). These mechanisms are briefly described below.

Impaired transport of antifolates via the RFC, either due to quantitative (decreased expression) and/or qualitative (increased Km and/or decreased Vmax) changes, has been established as an important and frequent mechanism of resistance to antifolates, both in vitro and in vivo (Fry and Jackson, 1986; Galivan, 1981; Jackman et al., 1986; McCormick et al., 1981; Niethammer and Jackson, 1975; Schuetz et al., 1988; Goldman et al., 1968). Recently, several studies with mouse (Tse and Moran, 1998; Tse et al., 1998; Zhao et al., 1998a; Zhao et al., 1998b; Zhao et al., 1999) and human leukemia cell lines displaying impaired antifolate transport have shown that the mechanism underlying these antifolate resistance phenotypes is mutations in the RFC gene (Gong et al., 1997; Jansen et al., 1998). Thus, single nucleotide changes and thereby single amino acid substitutions in the mouse or human RFC were identified in leukemic cell lines, which disrupted RFC expression or abolished antifolate transport (Brigle et al., 1995; Gong et al., 1997; Jansen et al., 1998). Importantly, some mutations primarily occurring in certain predicted transmembrane domains of the mouse RFC brought about a major loss of antifolate transport, while differentially preserving sufficient reduced folate and folic acid uptake to support cellular growth (Gong et al., 1997; Jansen et al., 1998; Tse and Moran, 1998; Tse et al., 1998; Zhao et al., 1998a; Zhao et al., 1998b; Zhao et al., 1999).

Impaired MTX transport has also been reported for childhood ALL as a common mechanism of intrinsic (Matherly, 1995) or acquired drug resistance (Trippett et al., 1992), however, no identification of specific mutations in the RFC gene were correlated therewith. It will be appreciated in this respect that a major difference exists between mutations acquired in vivo and those obtained in vitro, as is evident from work done in other gene systems, such as the p53 gene (Giglia et al., 1998).

Transport-related resistance may also occur when transport systems other than the RFC are expressed at the cell surface, e.g., membrane-associated Folate Binding Protein (mFBP), which has a lower affinity for MTX when compared to natural folates (Jansen et al., 1989).

Overproduction of target enzymes, due to either gene amplification, increased transcription, or increased translation, is a frequently occurring mechanism of resistance to antifolate anticancer drugs (Schimke 1984; Schimke 1988), most prevalent in cell lines. The most frequent mechanism of resistance to MTX is DHFR gene amplification after stepwise selection with MTX (Bertino, 1993; Schimke 1984; Schimke 1988), in cultured cells (Dedhar et al., 1985; Ohnuma et al., 1985; Van der Laan et al., 1991), and, to a much lesser extent, in clinical samples from patients treated with MTX-containing chemotherapy (Li et al., 1982; Curt et al., 1983; Carman et al; 1984; Matherly and Angeles 1993; Goker et al., 1995). Gene amplification in vitro is very common after stepwise exposure to increasing MTX concentrations. Similarly, cultured cells that are exposed to TS inhibitors (e.g., CB3717, ZD1694) may also develop resistance to these drugs by overproduction of TS (Jackman et al., 1986; O'Connor et al., 1992). This overproduction can be a result of gene amplification, increased levels of mRNA and/or increased translation (Jackman et al., 1995; Freemantle et al., 1995). The enzyme responsible for the breakdown of (anti)folate polyglutamates; folylpoly γ-glutamate hydrolase (FPGH), can also become overexpressed as a result of drug treatment. This has been described for H35 hepatoma cells, made resistant to DDATHF, by gradually increasing the drug concentration in the culture medium (Rhee et al., 1993).

Catalytically altered target enzymes for antifolates, such as the enzyme DHFR, can either have a decreased affinity for MTX (Thillet et al., 1988), or altered kinetic properties with regard to their dihydrofolate substrates (Jackson and Niethammer, 1977; Dedhar and Goldie, 1985). Changes in the enzyme kinetics are often caused by point mutations in the gene, which sometimes lead to a dramatic loss of substrate (e.g. antifolate) binding affinity. An altered DHFR in mutant 3T6 fibroblasts has been reported which displays an affinity for MTX that is 270-fold lower than wild type DHFR (Haber and Schimke, 1984).

Decreased polyglutamylation of antifolates, through decreased FPGS expression, has been associated with inherent or acquired resistance to MTX, in vitro and in vivo, similarly to ZD1694. Tumor cell resistance as a result of decreased FPGS activity often occurs after short-term incubations with high concentrations of antifolates. Decreased polyglutamylation can also correlate with resistance to 5-fluorouracil, which is explained by the impaired polyglutamylation of 5, 10-methylenetetrahydrofolate, the folate cofactor which forms a ternary complex with TS and 5-fluorodeoxyuridylate.

The Reduced Folate Carrier (RFC):

In a wide variety of human and rodent tumor cells, an active carrier-mediated transport system has been identified for the cellular uptake of reduced folate cofactors. The natural substrate for this reduced folate carrier (RFC) is the circulating plasma folate, 5-methyltetrahydrofolate. Although reduced folates are the preferential substrate for RFC, various hydrophilic antifolates including MTX, ZD1694, GW1843, edatrexate, PT523, ZD9331, DDATHF, aminopterin, and MTA are transported efficiently via this carrier. RFC expression has been observed in fetal tissues, suggesting that this expression is a fetal membrane property that is differentially expressed among normal and neoplastic cells. Efficient transport via the RFC appears to be essential for cellular folate homeostasis and antifolate drug sensitivity, whereas impaired RFC-transport may be a mechanism for intrinsic or acquired resistance to antifolate drugs.

The transport kinetics of MTX in murine leukemia cells was first characterized by Kessell et al. in 1965 and by Goldman in 1968. Similar kinetic properties were described later on, for several other tumor cells derived from various tissues. The RFC exhibits saturable transport (Kₘ=1−3 μM) for 5-substituted reduced folates, such as 5-formyltetrahydrofolate (leucovorin, LCV), 5-methyltetrahydrofolate (5—CH₃—THF), and MTX (Kₘ=3−26 μM). However, folic acid is poorly transported via the RFC (Kₘ200−400 μM). The transport Vmax for MTX measured at 37° C. varies between 1.0 and 12.2 pmol/min/mg protein, depending on the tumor cell line. RFC-transport is pH-, energy- and temperature-dependent (Q₁₀ 27-37° C.: 6-8) and is insensitive to the Na+/K+-ATPase inhibitor, ouabain.

A characteristic feature of RFC-mediated transport of MTX is that it can be inhibited by a variety of structurally (un)related organic and inorganic anions, including, for example, phosphate, chloride, sulphate, bicarbonate, probenecid, citrate, NADP, NAD, AMP and ADP. Furthermore, preloading intact cells with leucovorin, or loading plasma membrane vesicles with phosphate, both (trans)stimulated the uptake of MTX. These observations supported the hypothesis that anion gradients may serve as a driving force for the exchange uptake of the divalent anion MTX. Although this hypothesis per se is an attractive one (since it may guarantee electroneutrality in exchange), a number of issues have not yet been clarified. For example, thus far a direct exchange of an intracellular anion and an extracellular anion has not been clearly demonstrated. Furthermore, anion concentrations required to trans-stimulate/inhibit MTX transport often exceed physiological levels, thus questioning the physiological relevance of this observation. Finally, unlike in artificial anion-deficient buffer systems, trans-stimulation effects in physiological (i.e. chloride) containing buffer systems are significantly lower. Alternative explanations for these observations consider that either anion gradients may influence the mobility of the carrier across the membrane, or that anions influence the ionization status of MTX, thereby facilitating its uptake.

RFC Genetics and Biochemistry:

The molecular weight (MW) of the RFC protein has been determined using N-hydroxy-succinimide (NHS) esters of radiolabelled folates, biotin-conjugates of folates or photo-activated folate analogs. More recent reports described the MW of RFC in rodent cells to be of approximately 46-48 kDa. In human leukemic cells (CCRF-CEM, K562 and HL60 cells) the MW of the RFC is substantially higher (80-120 kDa) due to an extensive N-linked glycosylation. The RFC in K562 cells contains both N- and O-linked glycosylated residues. Inhibition of glycosylation by tunicamycin resulted in a reduction of transport (Vₘₐₓ) of approximately 30%, concomitant with a decrease in the number of carrier molecules at the cell surface, whereas the transport Km was unchanged.

Recent studies reported the isolation of a cDNA clone from murine L1210 leukemia and CHO cells that was able to complement MTX transport in RFC transport defective cells, and could restore MTX sensitivity. The mouse protein encoded by the RFC1 cDNA has a predicted MW of 58 kDa and contains one consensus signal for N-glycosylation. On the basis of hydropathy plots, the protein has 12 transmembrane domains (TMDs) and exhibits similarity with the 55 kDa human GLUT1 glucose transporter. Three other reports described the isolation of the human homologue of the RFC1 protein from placenta, HSC93 and K562 leukemic cell cDNA libraries, respectively. The human RFC is an integral plasma membrane protein having 591 amino acids, it is similarly predicted to contain 12 TMD and has a short N-terminus and a long C-terminus, both of which are hydrophilic and reside within the cytoplasm (Ferguson and Flintoff, 1999; Drori 2000).

The predicted core MW of the human RFC1 protein is 64 kDa, the size of the principal mRNA transcript is 2.7-3.1 kb, and a single consensus signal for N-glycosylation is present. However, the human RFC1 undergoes extensive glycosylation, resulting in a broadly migrating protein with a molecular mass of 70-120 kDa. The amino acid sequence of the human RFC1 protein has 51-70% homology with the hamster and murine RFC1. As with rodent RFC1, transfection of MTX transport deficient cells with the human RFC1 cDNA restored MTX uptake and MTX sensitivity. Lefebvre et al. and Brigle et al. demonstrated that, at least in cell lines, mutations in the RFC1 gene can provide a molecular basis for transport-related MTX resistance.

Biodistribution:

It has been shown that the activity of the RFC is an important clinical parameter and a potential prognostic factor for successful MTX treatment of childhood ALL. Determination of the tissue expression levels of the RFC might have clinical relevance for antifolate therapy. On the basis of transport kinetic studies, RFC expression was demonstrated in some normal mammalian cells (e.g., mouse small intestine, rat hepatocytes and rabbit kidney) and in various rodent and human derived tumor cells including leukemia, sarcomas, hepatomas and carcinomas of breast, lung and ovary. Matherly et al. were able to determine the human tissue distribution of RFC by immunohistochemistry. These studies also showed that RFC was present in many cells and tissues, both normal and neoplastic. High RFC expression was detected in prostate, testis, liver, and adrenal sections. In tissue sections from primary human tumors, the RFC was detected in melanoma, lymphoma, and astrocytoma sections and in prostatic, pancreatic, gastric, thyroid, breast, ovarian, colon, renal, hepatocellular, and lung carcinomas.

As has already been said above, although the involvement of RFC in reduced folate and antifolate compounds uptake is well studied and further, although mutations in rodent RFC gene and a single mutation in human RFC gene which alter such uptake were detected in rodent and human cell lines, no evidence has been so far provided to the effect that a similar mechanism of altered uptake characterizes tumor cells in vivo. While reducing the present invention to practice, as is further detailed and exemplified in the following sections, it was found that at least in some cases RFC mutations are responsible for such altered uptake in vivo. These findings have immense therapeutic implications in selection of appropriate chemotherapy and monitoring the cancer patient through and between chemotherapy sessions.

Mammalian cells cannot synthesize folates and therefore must rely on their own uptake from exogenous sources (1). As detailed above, the reduced folate carrier (RFC) is the major transport route for folates and hydrophilic antifolates including methotrexate (MTX). RFC functions as a bidirectional anion exchanger with high affinity for reduced folates and MTX. The latter is a folic acid antagonist which exerts its cytotoxic activity by a high affinity (K_{D}=1 pM) inhibition of the target enzyme DHFR, the key enzyme in tetrahydrofolate biosynthesis. This blockade leads to inhibition of tetrahydrofolate biosynthesis, inhibition of DNA replication and cell death.

Like natural folates, the various hydrophilic antifolates enter cells primarily via RFC. However, a more minor uptake pathway involves the membrane-anchored folic acid receptor (FR) α and β.

Once in the cell, and as is further detailed hereinabove, reduced folates as well as antifolates undergo a complex metabolism including addition of up to 10 glutamyl residues, a process known as polyglutamylation and which is catalyzed by the enzyme FPGS. Thus, FPGS which possesses an ATPase activity, catalyzes the conjugation of glutamyl residues to the γ-carboxyl of the glutamate residue of natural folates and hydrophilic antifolates. Polyglutamylation of natural reduced folates and glutamate-containing antifolate drugs renders them polyanionic and thereby results in their entrapment within the cells. However, the activity of FPGS can be counteracted by the antagonistic enzyme FPGH. This enzyme cleaves off the polyglutamyl tail and consequently brings the natural folate or antifolate to their original monoglutamate form. Thus, this system of antagonistic enzymes FPGS and FPGH maintains the necessary folate homeostasis. In the monoglutamate form (non-polyglutamylated), reduced folates and hydrophilic antifolate anticancer drugs are efficiently extruded by several ATP-driven exporters of the Multidrug Resistance Protein (MRP) family, namely MRP-1, -2 and -3. Thus, these members of the MRP family of transporters are an important component of folate homeostasis. Furthermore, the ability of these MRPs to extrude hydrophilic antifolate anticancer drugs confers upon cells anticancer drug resistance. In contrast, lipid-soluble antifolates (lacking the glutamate residue) including trimetrexate and piritrexim are extruded from multidrug resistant (MDR) cells by P-glycoprotein (Pgp). Pgp functions as an ATP-driven efflux transporter which pumps out of tumor cells a variety of hydrophobic anticancer drugs including anthracyclines, Vinca alkaloids, taxoids, epipodophyllotoxins and actinomycin D. Both Pgp and MRPs are members of the ABC (ATP-Binding Cassette) superfamily of transport ATPases.

Reduced folates serve primarily in the biosynthesis of purines. In this respect, apart from DHFR, the catalytic activity of two important enzymes, glycinamide ribonucleotide transformylase (GARTF), and 5-aminoimidzole 4-carboxamide ribonucleotide transformylase (AIRCARTF) is absolutely required for purine ring biosynthesis. Thus, GARTF and AIRCARFT are formyl transferases which utilize 10-formyltetrahydrofolate as the one-carbon donor for the biosynthesis of the purines, AMP and GMP.

Furthermore, reduced folates are also absolutely required for the biosynthesis of the pyrimidine, thymidylate (dTMP). Thus, the enzyme thymidylate synthase (TS) catalyzes the biosynthesis of thymidylate from uridylate using the natural folate cofactor, 5, 10-methylenetetrahydrofolate.

Mammalian cells absolutely rely on the de novo folate metabolic pathway for the biosynthesis of purine and pyrimidine nucleotides. In this respect, since the activity of these folate-dependent enzymes is absolutely required for the growth of replicating cells, many leading pharmaceutical companies have targeted these folate-dependent enzymes over the past two decades by rationally-designed novel and potent antifolates (see, Table 1, below). In this respect, Tomudex (Raltitrexed, ZD1694) was recently approved for use against advanced (i.e., metastatic) colorectal cancer. Tomudex is a high affinity inhibitor of TS. Like MTX, Tomudex is taken up efficiently by the RFC and within the cells it undergoes polyglutamylation and thereby increased retention.

However, the cytotoxicity of antifolates anticancer drugs like MTX and Tomudex in the clinical oncology setting can been abolished by the frequent emergence of drug resistance phenomena.

TABLE 1
Novel antifolates, their cellular targets, the pharmaceutical companies
and Institutions involved in their clinical evaluation

| | | Clinical | | |
|---|---|---|---|---|
| Antifolate | Target | Evaluation | Clinical Use | Company |
| Tomudex | TS | Approved | Colorectal Cancer | Zeneca |
| ZD9331 | TS | Phase II | — | Zeneca |
| MTA | TS, GARTF | Phase III | Colorectal, | Eli Lilly |
| DHFR | | | Pancreas, NSCLC | |
| | | | Breast, Head & | |
| | | | Neck | |
| | | | Genitourinary | |
| | | | Gynecologic | |
| GW1843 | TS | Phase II | — | Glaxo- |
| | | | | Wellcome |
| Piritrexim | DHFR | Phase III | — | Glaxo- |
| | | | | Wellcome |
| Trimetrexate | DHFR | Phase III | — | Warner- |
| | | | | Lambert |
| AG2034 | GARTF | Phase I/II | — | Agouron |
| AG337 | TS | Phase II | — | Agouron |
| AG377 | TS | Phase II | — | Agouron |
| DDATHF | GARTF | Phase I/II | — | Eli Lilly |
| PT523 | DHFR | Phase I | — | Dana- |
| | | | | Farber |
| Edatrexate | DHFR | Phase II | — | Sloan- |
| | | | | Kettering |

Although the involvement of these proteins in reduced folate and antifolate uptake and/or metabolism is well studied and further although mutations in the genes encoding same and which alter their activity were detected in various cell line systems, no evidence has been so far provided to the effect that a similar mechanism of altered uptake or metabolism characterizes tumor cells in vivo.

There is thus a widely recognized need for, and it would be highly advantageous to have, a method for and a kit for the assessment of the responsiveness of cancer patients to antifolate chemotherapy.

### SUMMARY OF THE INVENTION

According to one aspect of the present invention there is provided a method of assessing a responsiveness of a cancer patient to antifolate chemotherapy, the method comprising the step of searching for a mutation or mutations in a gene associated with folate metabolism or uptake in cells derived from the patient.

According to further features in preferred embodiments of the invention described below, the gene associated with folate metabolism or uptake is reduced folate carrier (RFC) gene.

According to still further features in the described preferred embodiments the gene associated with folate metabolism or uptake is selected from the group consisting of dihydrofolate reductase (DHFR) gene, folylpolyglutamate synthetase (FPGS) gene, folylpoly γ-glutamate hydrolase (FPGH) gene, glycinamide ribonucleotide transformylase (GARTF) gene, phosphoribosylamino-imidazole-carboxamide transformylase (AICARTF) gene, thymidylate synthase (TS) gene, P-glycoprotein (Pgp) gene, multidrug resistance protein 1 (MRP1) gene, multidrug resistance protein 2 (MRP2) gene and multidrug resistance protein 3 (MRP3) gene.

According to still further features in the described preferred embodiments the cells are tumor cells.

According to still further features in the described preferred embodiments the cells are non-tumor cells.

According to still further features in the described preferred embodiments, the method further comprising the step of evaluating an effect of mutation or mutations found in the gene on antifolate and reduced folate uptake or metabolism in tumor cells of the patient.

According to still further features in the described preferred embodiments the step of evaluating an effect of the mutation or mutations found in the gene on the antifolate and the reduced folate uptake or metabolism in the tumor cells of the patient is effected by previous knowledge regarding the mutation or mutations.

According to still further features in the described preferred embodiments the step of evaluating an effect of the mutation or mutations found in a reduced folate carrier (RFC) gene on the antifolate and the reduced folate uptake in the tumor cells of the patient is effected by evaluating the effect on uptake of the antifolate and the reduced folate by cells expressing a mutated reduced folate carrier protein encoded by a polynucleotide harboring the mutation or mutations.

According to another aspect of the present invention there is provided a method of assessing a responsiveness of tumor cells to antifolate chemotherapy, the method comprising the step of searching for a mutation or mutations a gene associated with folate uptake or metabolism in the tumor cells.

According to further features in preferred embodiments of the invention described below, the method further comprising the step of evaluating an effect of mutation or mutations found in the gene on antifolate and reduced folate uptake or metabolism in the tumor cells.

According to still further features in the described preferred embodiments the step of evaluating an effect of the mutation or mutations found in the gene on the antifolate and the reduced folate uptake or metabolism in the tumor cells is effected by previous knowledge regarding the mutation or mutations.

According to still further features in the described preferred embodiments the step of evaluating an effect of the mutation or mutations found in a reduced folate carrier (RFC) gene on the antifolate and the reduced folate uptake in the tumor cells is effected by evaluating the effect on uptake of the antifolate and the reduced folate by cells expressing a mutated reduced folate carrier protein encoded by a polynucleotide harboring the mutation or mutations.

According to still further features in the described preferred embodiments the step of searching for the mutation or mutations in the reduced folate carrier (RFC) gene in the cells derived from the patient is effected by a single strand conformational polymorphism (SSCP) technique.

According to still further features in the described preferred embodiments the single strand conformational polymorphism technique is selected from the group consisting of cDNA-SSCP and genomic DNA-SSCP.

According to still further features in the described preferred embodiments the step of searching for the mutation or mutations in the reduced folate carrier (RFC) gene in the cells derived from the patient is effected, at least in part, by a technique selected from the group consisting of nucleic acid sequencing, polymerase chain reaction, ligase chain reaction, self-sustained synthetic reaction, Qβ-replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

According to yet another aspect of the present invention there is provided a kit for assessing a responsiveness of a cancer patient to antifolate chemotherapy, the kit comprising a holder for holding at least one container containing oligonucleotides capable of amplifying at least one fragment of a gene associated with folate uptake or metabolism.

According to further features in preferred embodiments of the invention described below, the kit further comprising a container containing a DNA polymerase enzyme.

According to still further features in the described preferred embodiments the kit further comprising a container containing a reverse transcriptase enzyme.

According to still further features in the described preferred embodiments the kit further comprising a container containing a mixture of dNTPs.

According to still further features in the described preferred embodiments the kit further comprising a container containing a concentrated polymerase chain reaction buffer.

According to still further features in the described preferred embodiments the oligonucleotides are selected from the group of oligonucleotides identified by SEQ ID NOs:1-20.

According to still further features in the described preferred embodiments at least one of the oligonucleotides includes at least one nucleotide analog.

According to still further features in the described preferred embodiments at least one of the oligonucleotides includes a labeling moiety.

According to still further features in the described preferred embodiments the kit further comprising at least one precast gel for executing single strand conformational polymorphism.

According to still further features in the described preferred embodiments the kit further comprising at least one container containing reagents for detection of electrophoresed nucleic acids.

The present invention successfully addresses the shortcomings of the presently known configurations by providing a method of and a kit for assessing the responsiveness of cancer patients to antifolate chemotherapy.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show details of the invention in more detail than is necessary for a fundamental understanding of the invention, the description taken with the drawings making apparent to those skilled in the art how the several forms of the invention may be embodied in practice.

In the drawings:

FIG. 1 illustrates the chemical structures of folic acid, the reduced folate 5-CHO-tetrahydrofolate (leucovorin) and MTX.

FIG. 2 illustrates the major pathwvays of folate metabolism in the cytosol and in mitochondria of eukaryotes.

FIG. 3 is a flow diagram illustrating a protocol of a genomic PCR-SSCP assay for screening of mutations in the human RFC gene according to the present invention.

FIG. 4a is a genomic PCR-SSCP analysis of RFC exon 2 from parental CEM human leukemia cells, and their MTX transport-deficient CEM-MTX subline. Genomic DNA (0.5 μg) from CEM cells (lane a) and their MTX transport-deficient CEM-MTX subline (lane b) was PCR amplified in the presence of [³²P]dATP, using oligonucleotide primers targeting RFC exon 2. The PCR products were resolved by electrophoresis on 10-12.5% polyacrylamide gels containing glycerol. Bands with wild type electrophoretic mobility are denoted by arrows, whereas those with aberrant (i.e., mutant) electrophoretic mobility are marked by arrowheads.

FIG. 4b is a genomic PCR-SSCP analysis of RFC exon 3 from parental CEM human leukemia cells, and their MTX transport-deficient CEM-MTX subline. Genomic DNA (0.5 μg) from CEM cells (lane a) and their MTX transport-CEM-MTX subline (lane b) was PCR amplified in the presence of [³²P]dATP using intronic primers targeting RFC exon 3. The PCR products were resolved by electrophoresis on 10-12.5% polyacrylamide gels lacking glycerol. Bands with wild type electrophoretic mobility are denoted by arrows, whereas those with aberrant (i.e., mutant) electrophoretic mobility are marked by arrowheads.

FIG. 4c is a genomic PCR-SSCP analysis of RFC exon 4 from parental CEM human leukemia cells, and their MTX transport-deficient CEM-T subline. Genomic DNA (0.5 μg) from CEM cells (lane b) and their MTX transport-CEM-T subline (lane a) was PCR amplified in the presence of [³²P]dATP using intronic primers targeting RFC exon 4. The PCR products were resolved by electrophoresis on 10-12.5% polyacrylamide gels lacking glycerol. The band with aberrant (i.e., mutant) electrophoretic mobility is marked by an arrowhead.

FIG. 5 is a genomic PCR-SSCP analysis of RFC exons 3 to 6 from parental ZR-75 human breast carcinoma cells and their MTX transport-impaired MTX^{R}-ZR-75 subline. Genomic DNA from ZR-75 cells (lanes a, c, e, g, i, k and m) and their MTX transport-deficient MTX^{R}-ZR-75 human breast carcinoma subline (lanes b, d, f, h, j, l and n) was PCR amplified in the presence of [³²P]dATP, using oligonucleotide primers targeting exon 3 (lanes a-f), exon 4 (lanes g and h), exon 5 (lanes i and j) and exon 6 (lanes k-n) of the RFC gene. The PCR products were resolved by electrophoresis on 10% polyacrylamide gel. The band with aberrant electrophoretic mobility in MTX^{R}-ZR-75 cells is marked by an arrowhead and an asterisk (lane j).

FIG. 6a illustrates antifolate growth inhibition in parental CEM cells and their GW70 subline. Exponentially growing parental CEM (circles) and GW70 cells (squares) in folic acid-containing medium were exposed for 3 days to various concentrations of GW1843U89, after which viable cells were counted.

FIG. 6b illustrates growth inhibition in parental CEM cells and their GW70 subline. Exponentially growing parental CEM (circles) and GW70 cells (squares) in folic acid-containing medium were exposed for 3 days to various concentrations of MTX, after which viable cells were counted.

FIG. 6c illustrates folic acid growth requirement in parental CEM cells (circles) and their GW70 subline (squares). Cells grown for 1-2 weeks in GAT-containing folate-free medium were exposed for 3 days to various folic acid concentrations, after which viable cells were counted.

FIG. 7a illustrates transport of [³H]MTX into parental CEM cells and their GW70 subline. Exponentially growing parental CEM (open circles) and GW70 cells (solid circles) were washed twice with HBSS, adjusted to 2×10⁷ cells/ml and the transport of [³H]MTX was determined at an extracellular radiolabel concentration of 2 μM.

FIG. 7b illustrate transport of [³H]folic acid into parental CEM cells and their GW70 subline. Exponentially growing parental CEM (open circles) and GW70 cells (solid circles) were washed twice with HBSS, adjusted to 2×10⁷ cells/ml and the transport of [³H]folic acid was determined at an extracellular radiolabel concentration of 2 μM.

FIG. 8a demonstrates Southern blot analysis of the RFC gene and its expression in CEM, GW70 and GW70/LF cells. Genomic DNA (10 μg) was digested with EcoRI, fractionated on 0.8% agarose gel, transferred to a nylon membrane and hybridized with a [³²P] labeled human RFC cDNA probe. Lanes a, b, and c are, wild type CEM, GW70, and GW70/LF cells, respectively.

FIG. 8b demonstrates Northern blot analysis of the RFC gene and its expression in CEM, GW70, and GW70/LF cells. Total RNA (20 μg) was fractionated on a denaturing 1% agarose gel containing formaldehyde, blotted onto a nylon membrane and hybridized with a [³²P]labeled human RFC and β-actin cDNA probes. Lanes a, b, and c are wild type CEM, GW70, and GW70/LF cells, respectively.

FIG. 8c demonstrates Western blot analysis of the RFC gene and its expression in CEM, GW70, and GW70/LF cells. Proteins (50 μg) of plasma membrane-enriched fraction were resolved by electrophoresis on a 10% polyacrylamide gel, electroblotted onto a PVDF nylon membrane and reacted with a polyclonal serum anti-recombinant human RFC. Lanes a, b, and c are wild type CEM, GW70, and GW70/LF cells, respectively.

FIG. 9a illustrates a genomic PCR-SSCP analysis of RFC exon 2 from parental CEM cells and their transport-altered sublines, GW70 and GW70/LF. Genomic DNA from CEM cells, their sublines GW70, and GW70/LF (lanes a, b and c, respectively) was PCR amplified in the presence of [³²P]dATP using oligonucleotide primers targeting RFC exon 2. The PCR products were resolved by electrophoresis on pre-casted commercial 10% polyacrylamide SSCP gel (ExcelGel, Pharmacia). Bands with wild type electrophoretic mobility are denoted by thin lines, whereas those with aberrant electrophoretic mobility are marked by arrowheads.

FIG. 9b illustrates a genomic PCR-SSCP analysis of RFC exon 3 from parental CEM cells and their transport-altered sublines, GW70 and GW70/LF. Genomic DNA from CEM cells, their sublines GW70, and GW70/LF (lanes a, b and c, respectively) was PCR amplified in the presence of [³²P]dATP using oligonucleotide primers targeting RFC exon 3. The PCR products were resolved by electrophoresis on pre-casted commercial a 10% polyacrylamide SSCP gel (ExcelGel, Pharmacia). Bands with wild type electrophoretic mobility are denoted by arrows, whereas those with aberrant electrophoretic mobility are marked by arrowheads.

FIG. 9c is genomic PCR-SSCP analysis of RFC exons 2 from parental CEM cells and their transport-altered sublines, GW70 and GW70/LF. Genomic DNA from CEM cells, their sublines GW70, and GW70/LF (lanes a, b and c, respectively) was PCR amplified in the presence of [³²P]dATP using oligonucleotide primers targeting RFC exon 2. The PCR products were resolved by electrophoresis on laboratory-made glycerol containing gel. Bands with wild type electrophoretic mobility are denoted by arrows, whereas those with aberrant electrophoretic mobility are marked by arrowheads. Note that in exons 2 and 4 (FIGS. 7a-b) normal bands (arrows) were observed with wild type CEM cells, whereas GW70 and GW70/LF cells (lane a, b and c, respectively) were so altered that these bands could no longer be detected.

FIG. 9d illustrates a genomic PCR-SSCP analysis of RFC exons 3 from parental CEM cells and their transport-altered sublines, GW70 and GW70/LF. Genomic DNA from CEM cells, their sublines GW70, and GW70/LF (lanes a, b and c, respectively) was PCR amplified in the presence of [³²P]dATP using oligonucleotide primers targeting RFC exon 3. The PCR products were resolved by electrophoresis on laboratory-made glycerol lacking gel. Bands with wild type electrophoretic mobility are denoted by arrows, whereas those with aberrant electrophoretic mobility are marked by arrowheads.

FIG. 10a illustrates a genomic PCR-SSCP analysis of RFC exon 2 with DNA from an ALL patient (M1), blood from a healthy donor, and CEM-MTX cells. Genomic DNA from blood of a healthy donor (lane a), a B-precursor ALL tumor specimen (M1) obtained at diagnosis (lane b), and CEM-MTX cells (lane c), was PCR amplified using primers targeting RFC exon 2. The PCR products were resolved by electrophoresis on laboratory-made glycerol lacking gel. Bands with wild type electrophoretic mobility are denoted by arrows, whereas those with aberrant electrophoretic mobility are marked by arrowheads.

FIG. 10b illustrates a genomic PCR-SSCP analysis of RFC exon 2 with DNA from an ALL patient (M1), blood from a healthy donor, and CEM-MTX cells. Genomic DNA from blood of a healthy donor (lanes a and c) and a B-precursor ALL tumor specimen (M1) obtained at diagnosis (lane b), was PCR amplified using primers targeting RFC exon 2. The PCR products were resolved by electrophoresis on pre-casted commercial 10% polyacrylamide SSCP gel (ExcelGel, Pharmacia). Bands with wild type electrophoretic mobility are denoted by thin lines, whereas those with aberrant electrophoretic mobility are marked by arrowheads the band from M1 with altered mobility is also denoted by an asterisk (lane b).

FIG. 10c illustrates a genomic PCR-SSCP analysis of RFC exon 6 with DNA from two ALL patients (J7 and J11), blood from a healthy donor, and CCRF-CEM cells. Genomic DNA from B-precursor ALL tumor specimens (J7, harboring the Asp522Asn mutation, and J11, apparently lacking any mutations) obtained at diagnosis (lanes a and b, respectively), blood from a healthy donor (lane c) and human CCRF-CEM cell line (lane d) was PCR amplified using primers targeting RFC exon 6. The PCR products were resolved by electrophoresis on pre-casted commercial 10% polyacrylamide SSCP gel (ExcelGel, Pharmacia). Arrow indicates bands with wild type electrophoretic mobility, whereas the lower band with aberrant electrophoretic mobility present only in J7 is marked by an arrowhead.

FIG. 10d illustrates MTX accumulation in blast cells from pre-B ALL patient J7 harboring the mutant Asp522Asn RFC and from common/pre-B ALL patients that do not show mutations in the RFC gene. Isolated leukemia cells (5×10⁶ cells) from patient J7 at diagnosis or from various leukemia patients that are apparently devoid of RFC gene mutations as judged from the gPCR-SSCP analysis were assayed for [³H]MTX uptake as detailed in the Examples section that follows. The average [³H]MTX accumulation for 15 leukemia patients with intact uptake was: 0.78 pmol/10⁷ cells/min, whereas that of J7 cells was only: 0.28 pmol/10⁷ cells/min. Data shown are the means of two independent experiments in which the variation did not exceed 15%.

FIGS. 11a-d are polymorphisms of G/A at nucleotide 174 (Arg/His 27) in exon 2 in the human RFC, observed in 30 individuals, as revealed by the genomic PCR-SSCP assay. Thirty DNA samples from both healthy individuals, as well as ALL patients, were PCR amplified using RFC exon 2-specific primers and the products were analyzed by electrophoresis on lab-casted (FIGS. 11a-c), or commercial SSCP gels (FIG. 11d). Three equally distributed polymorphic groups at nucleotide position 174 exon 2 were: one group of individuals was homozygous for G174, thus containing Arg 27 (arrowheads), another group was homozygous for A174, thus displaying the His 27 variant (FIG. 11d, arrows), and the remaining group was heterozygous, namely containing G and A at nucleotide 174, thus expressing two RFC species, one with Arg 27, and another with His 27 (arrowheads and arrows).

FIG. 12 is a perspective view of a kit according to the present invention. FIG. 13 is a schematic depiction of human RFC missense mutations identified by the genomic PCR-SSCP assay in antifolate-resistant transport-deficient leukemia cell lines and childhood ALL specimens. The predicted secondary structure of the human RFC is based on the hydropathy analysis of Prasad et al., (1995). Mutated amino acid residues are shown as solid circles whereas the Arg/His polymorphism at amino acid 27 is shown as a gray circle.

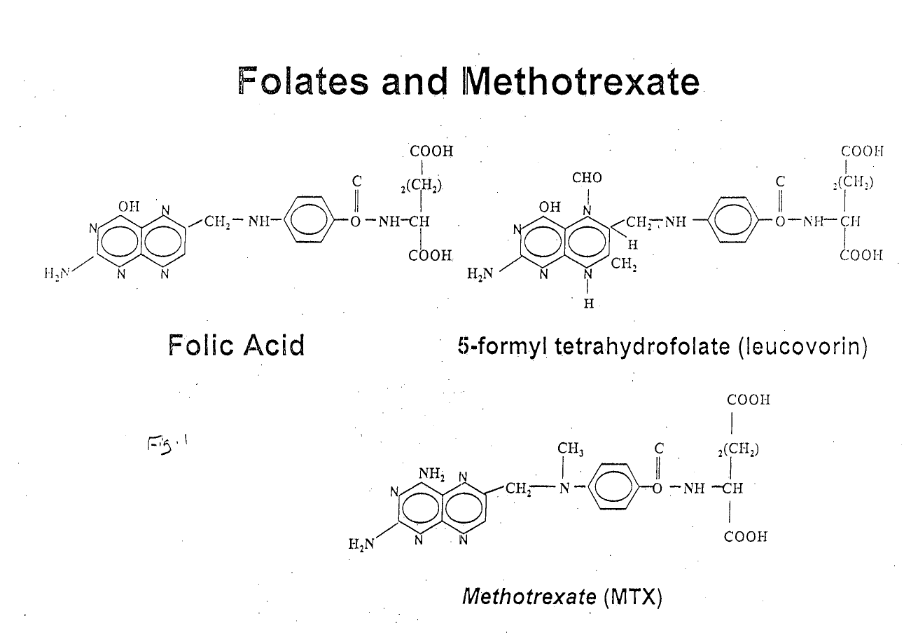

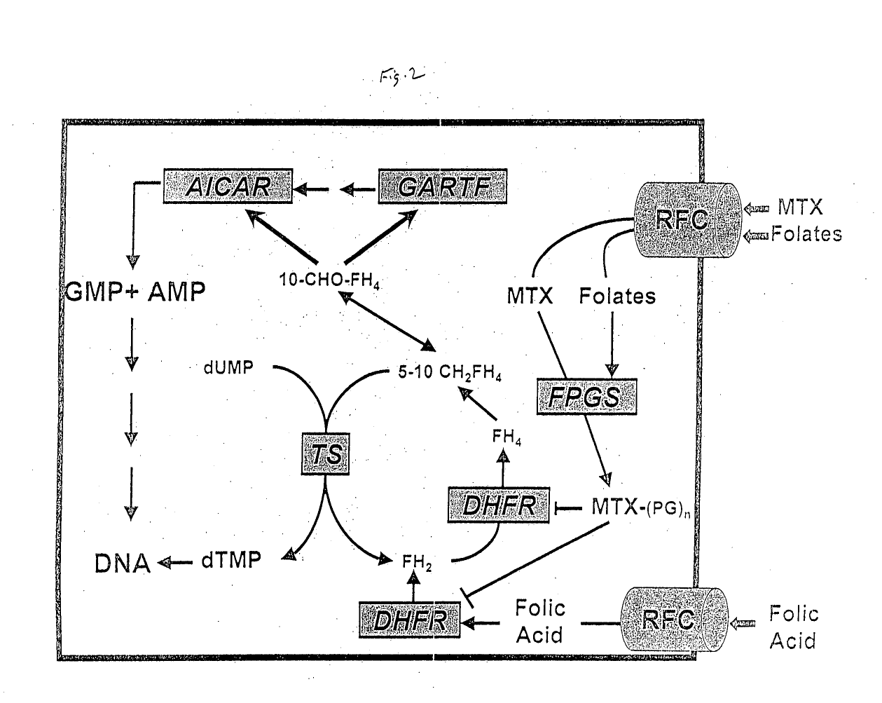

### DESCRIPTION OF THE PREFERRED EMBODIMENTS

The present invention is of a method and a kit which can be used for assessing the responsiveness of cancer patients to antifolate chemotherapy. Specifically, the present invention can be used to detect mutation or mutations in the reduced folate carrier (RFC) gene in cells, either cancer or non-tumor cells, of cancer patient, to thereby evaluate the responsiveness of the cancer patients to antifolate chemotherapy.

The principles and operation of a according to the present invention may be better understood with reference to the drawings and accompanying descriptions.

Before explaining at least one embodiment of the invention in detail, it is to be understood that the invention is not limited in its application to the details of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While reducing the present invention to practice, a novel genomic PCR-single strand conformational polymorphism (SSCP) assay for the rapid identification of reduced folate carrier (RFC) mutations in multiple antifolate-resistant cell lines and tumor specimens has been developed and tested. Polymerase chain reaction (PCR)-single strand conformational polymorphism (SSCP) analysis (Jaekel et al., 1998) has been applied extensively for the detection of sequence variations (i.e., mutations and polymorphisms) in multiple samples in a wide variety of genes for example p53 (Giglia et al., 1998), Ki-Ras (Bolag et al., 1995), thymidylate synthase (Tony et al., 1998), and the transforming growth factor β receptor type II (Lynch et al., 1998). Using oligonucleotide primer pairs spanning the entire human RFC coding region, genomic DNA was PCR amplified and subjected to SSCP analysis. Amplified DNA products with aberrant electrophoretic mobility were sequenced and the role of the observed mutation on (anti)folate transport, antifolate resistance and folate growth requirement was determined after its stable transfection in transport-deficient cells. Human CCRF-CEM leukemia GW70 cells established by stepwise selection with GW1843, a novel thymidylate synthase inhibitor and thus far, the best transport substrate for the human RFC, displayed a high level resistance to GW1843 and to methotrexate (MTX), but surprisingly had a decreased folic acid growth requirement as compared to parental CEM cells. These alterations were associated with a 6-fold decreased affinity of RFC for GW1843, an 11-fold and 3-fold increased affinity for folic acid and 5-methyltetrahydrofolate, respectively. As these results were consistent with a structurally altered RFC, DNA from GW70 cells was examined by the SSCP assay followed by sequencing. RFC from GW70 cells contained several alterations: (i) three single nucleotide changes resulting in the following amino acid substitutions: Val29Leu, Glu45Lys, and Ser46Ile; (ii) upon adaptation of GW70 cells to grow on low folic acid, a GW70-LF cell line was obtained which acquired an additional Asp88His mutation; and (iii) whereas parental CEM cells were heterozygous, thus containing both Arg 27 and His 27, GW70 cells were homozygous for Arg 27. Analysis of multiple blood sample DNA confirmed the polymorphic Arg/His variation at amino acid 27.

The genomic PCR-SSCP assay was also used to screen childhood acute lymphoblastic leukemia (ALL) specimens. One B-precursor ALL patient contained at diagnosis a His56Asp substitution in the RFC, and was also homozygous for His 27. This patient relapsed 15 months later. Transfection studies with the mutant His27/His56 RFC cDNA demonstrated both MTX resistance and a notable decrease in the folic acid and leucovorin growth requirements as compared to native RFC transfectant. Another substitution, Asp522Asn was detected by the PCR-SSCP assay of the invention in blood cells DNA of a B-precursor ALL patient (J7) who was completely resistant to chemotherapy, and subsequently died of his illness. MTX uptake studies comparing blast cells from patient J7 to antifolate sensitive cells revealed significantly impaired MTX uptake in the ALL patients cells. These results demonstrate the usefulness of this SSCP assay in the rapid screening of tumor cells and patient specimens for multiple RFC mutations, some of which disrupt antifolate transport while preserving natural folate uptake. This constitutes the first documentation of RFC mutations in tumor specimens using a high throughput screening method of the entire RFC gene.

The assessment of human RFC mutations by a genomic PCR-SSCP assay has several advantages over the recently used reverse transcriptase (RT)-PCP followed by sequencing (Jansen et al., 1998). First, it is a high throughput screening assay, well suited for the simultaneous handling of multiple genomic DNA samples derived from different sources including blood, tumor cell lines and tumor specimens. Second, it utilizes genomic DNA, thus avoiding the more difficult handling of RNA extraction, especially from tumor specimens, when using RT-PCR (Jansen et al., 1998). Furthermore, the SSCP assay utilizes genomic DNA which is a stable nucleic acid and also requires only submicrogram DNA quantities per analysis, which translates into a small number of cells (˜10⁵), a key advantage upon analysis of clinical specimens. Third, it is an inexpensive, facile, rapid, and efficient assay as a small DNA fragment (≦300 bp) with an electrophoretic mobility change is identified on a single sequencing gel. This stands in complete contradistinction with the recently used RT-PCR and sequencing of the entire RFC cDNA which is, of course, labor-intensive, time-consuming, expensive, and above all, far from being suitable for the handling of a large number of samples. The assay can be entirely performed in the absence of radiolabeled nucleotides using silver staining of the electrophoretically resolved genomic PCR products. Furthermore, the assay can be performed with commercially available, ready to use, SSCP gels.

The genomic PCR-SSCP assay proved effective in detecting different genetic alterations occurring in the human RFC gene including: (i) single nucleotide substitutions within an exon: for example, a G to C transition at nucleotide position 227 (exon 2), and a C to T change at nucleotide 352 (exon 3), both of which occur in CEM-MTX cells (see Table 4 in the examples section that follows; (ii) multiple nucleotide changes within an exon: GW70 cells contained three single nucleotide substitutions in exon 2 (Table 4); (iii) deletion: MTX^{R}—ZR-75 breast carcinoma cells with a severe MTX transport impairment contained a 5 bp deletion in exon 4, thus leading to a frame-shift and premature translation termination (Table 4); (iv) insertion: CEM-MTX cells with an MTX transport impairment contained a G inserted at nucleotide 1139 of exon 4, causing frameshift and premature translation termination Table 4); (v) polymorphism: the SSCP assay identified three equally distributed polymorphic variants at nucleotide 174 in exon 2; one third of the individuals were homozygous for G174, thus containing Arg 27, the other third was homozygous for A174, thus displaying His 27, and the remaining were heterozygous namely containing G and A at nucleotide 174, thus expressing two RFC species, one with Arg 27, and another with His 27.

This study constitutes the first report identifying a mutation in the RFC gene in a tumor (ALL) specimen. In this respect, a G to C mutation at nucleotide 261 in exon 2 was identified in a patient (M1) diagnosed with B-precursor ALL. This patient was further found to be homozygous for the polymorphic G174 in the same exon 2; these genetic alterations residing on the same allele, resulted in the generation of an RFC with His 27 and His 56. Transfection of the mutant RFC cDNA harboring these two mutations into transport-deficient mouse leukemia cells conferred MTX-resistance, when compared to a control transfection with the native RFC cDNA (with Arg 27). Surprisingly, the double-mutant RFC cDNA induced a simultaneous decrease in the folic acid and leucovorin growth requirements, as compared to the native transfectant. Thus, although the patient (M1) DNA sample was obtained at diagnosis and thus prior to antifolate-containing chemotherapy, pre-existing mutations occurred in the RFC gene which conferred upon these B-precursor ALL cells both MTX resistance and an increased ability to take up the folate cofactors leucovorin and folic acid. It is important to note that this patient had a fatal relapse 15 months after diagnosis. Using the same PCR-SSCP screening protocol, a second B-precursor ALL patient (J7) was found to harbor an Asp-Asn substitution in the C-terminus region of exon 6. Isolated blast cells from this patient demonstrated severely impaired MTX uptake, consistent with the complete and ultimately fatal resistance to chemotherapy observed clinically. These findings further suggest that RFC mutations need not be exclusively provoked by antifolate-containing chemotherapy, and some pre-existing mutations and/or genetic RFC variants may exist in tumor cell populations which may gain a growth advantage, thereby undergo clonal expansion upon treatment with antifolate anticancer drugs. This clearly raises the need for RFC mutation screening of tumor specimens not only after the chemotherapeutic treatment but also at diagnosis.

A major perplexing question in RFC-mediated resistance to various antifolates has been how transport defects can, on the one hand, abolish antifolate uptake, and on the other hand, have little effect on the growth requirement for the structurally homologous natural folate cofactors (e.g., leucovorin and folic acid). Several possibilities exist which may explain this dilemma. First, an RFC-independent route exists which serves as an alternative folate uptake pathway. As such, this alternative folate cofactor transport route could readily compensate for the loss of the major pathway, i.e., RFC, when mutations occurring in the latter disrupt both antifolate and folate cofactor uptake. Two independent routes that are distinct from RFC could comply with this alternative pathway paradigm: the membrane folate receptor (Antony, 1992; Wang et al., 1992; Brigle et al., 1994; Westerhof et al., 1995), and the low pH folate transporter (Henderson and Strauss, 1990; Kumar et al., 1997; Assaraf et al., 1998; Sierra et al., 1997). The latter mechanism gains experimental support from MTX-resistant (MTX^{r}A) mouse leukemia cells, which lost RFC transport activity for both MTX and natural folates, due to a mutation which disrupts carrier mobility (Brigle et al., 1995). Surprisingly these cells retained wild type uptake of folate cofactors via the low pH folate transporter (Jansen et al., 1997; Sierra and Goldman, 1998). Second, mutations could occur in the RFC which may differentially impair antifolate transport but preserve folate cofactor uptake, thereby allowing for cells to survive under antifolate cytotoxic pressure. Indeed, such a common theme clearly emerges from the following observations. First, in the present study, human leukemia GW70 cells, which display a high level resistance to the potent thymidylate synthase inhibitor, GW1843U89, had an impaired antifolate transport largely due to a marked fall in the affinity of RFC for this drug, and a small decrease in the drug transport Vₘₐₓ. At the same time, these GW70 cells displayed a dramatic increase in the affinity of RFC for folic acid, as well as a prominently increased affinity for leucovorin and 5-methyltetrahydrofolate. second, the severely transport-defective, MTX-resistant, human leukemia CEM-MTX cells contained a Lys for Glu substitution at amino acid 45 of the RFC, thereby rendering it a poor MTX transporter, but at the same time an excellent folic acid and leucovorin transporter, due to a dramatic increase in the transport affinity for both folic acid and leucovorin (Jansen et al., 1998). Third, consistently, recent studies with transport deficient MTX-resistant murine leukemia cells were found to contain the very same Glu 45 Lys mutation present in human CEM-MTX cells (Zhao et al., 1998). Consequently, this resulted in a markedly impaired MTX transport while preserving folic acid and leucovorin uptake due to a significant loss of carrier affinity for MTX, while gaining a marked increase in the transport affinity for folic acid. Fourth, further studies by Zhao et al., (1998) showed that another adjacent Ser46Asn substitution in a MTX-resistant murine L1210 cell line had a disproportional loss of MTX transport activity while selectively preserving sufficient leucovorin and 5-methyltetrahydrofolate uptake to meet their folate growth requirements. Fifth, independent studies by Tse et al. (Tse and Moran, 1998; Tse et al., 1998) with DDATHF-resistant murine leukemia cells identified two mutations in the TMD1 and TMD3, which resulted in a dramatic increase of this structurally altered RFC for folic acid. Thus, antifolate-resistant human and mouse leukemia cell lines shared in common the marked loss of antifolate transport while preserving natural folate cofactor uptake, thereby allowing them to meet their folate growth requirements under antifolate-selective pressure.

Recently, the laboratory of Goldman has initiated studies that characterize the relationship between structure and (anti)folate transport of the murine RFC using chemical mutagens aimed at generating a broad spectrum of mutations within the carrier (Zhao et al., 1998a; Zhao et al., 1998b; Zhao et al., 1999). Several selective pressures have been applied including leucovorin or folic acid as the sole folate source in order to mimic the specific conditions occurring in vivo and in vitro in order to generate a diversity of structural and thus functional alterations which delineate the RFC regions, that are key determinants for substrate binding and/or carrier mobility. Using this approach 30 different clonal MTX-resistant cell lines were characterized, the vast majority of which harbored single amino acid substitutions within a TMD (Zhao et al., 1999). Among these TMD-associated mutations, TMD1 appeared to be a mutation cluster region and was therefore proposed to play a key role in determining the functional properties of the RFC (Zhao et al., 1999). A strong support to this notion emerges from the SSCP screening described herein, in which the mutations altering (anti)folate transport activity in the human RFC from antifolate-resistant cells lines almost exclusively map to TMD1 (Table 3). Interestingly however, it was found that an RFC mutation (Asp56His) occurring in an ALL patient (M1) prior to chemotherapy mapped to putative loop 1 (i.e., connecting TMD1 and TMD2) predicted to reside in the extracellular milieu. This Asp56His mutation itself has no affect either on MTX sensitivity or folate growth requirement. However, when simultaneously present (as found in the M1 ALL patient) with the polymorphic N-terminal His 27 (predicted to reside in the cytosol), an RFC is generated that confers MTX resistance and at the same time allows for increased folate cofactor accumulation. These results indicate that apart from the TMDs, extramembranal RFC segments, such as loop 1, may play a contributing role in substrate binding and/or carrier mobility. Supportive evidence to this suggestion can be found in two tumor cell lines, in which a single amino acid substitution in a putative loop 7 of the mouse RFC (also predicted to reside extracellularly) was associated with a 3-fold difference in the influx Kₘ (Roy et al., 1998). Furthermore, site-directed mutagenesis studies with the external loops IV, V, and VI of the mammalian brain γ-aminobutyric acid transporter indicated that these short extramembranal loops presumably form a pocket, in which the substrate binds to this transporter (Roy et al., 1998).

The results with the mutations in the RFC establish that qualitative (i.e., mutations) and/or quantitative alterations in the variety of folate-dependent enzymes and transporters which are further detailed in the Background section above may lead to antifolate anticancer drug resistance.

Table 2 below summarizes the alterations that will bring about antifolate anticancer drug resistance in tumor cells:

TABLE 2
Qualitative and quantitative alterations which
cause antifolate anticancer drug resistance

The Alteration

| Resistance Protein | Mutation | Quantitative Change |
|---|---|---|
| DHFR | + | Increased expression |
| FPGS | + | Decreased expression |
| FPGH | − | Increased expression |
| GARTF | + | Increased expression |
| AICARTF | + | Increased expression |
| TS | + | Increased expression |
| Pgp | − | Increased expression |
| MRP 1-3 | + | Increased and decreased |
| | | expression* |

*Increased MRP1, -2 or -3 expression will enhance hydrophilic antifolate efflux and thus bring about antifolate anticancer drug resistance. On the other hand, decreased MRP1, -2 or -3 expression will decrease the efflux of natural folates and thereby expand the intracellular folate pool. Consequently, this will bring about a competitive negation of antifolate anticancer drug cytotoxicity thereby resulting in drug resistance.

Thus, mutations in DHFR which disrupt antifolate binding, but at the same time, retain sufficient folate substrate (i.e., dihydrofolic acid) binding and catalytic activity (dihydrofolic acid reduction) would result in anti-folate drug resistance.

Increased DHFR activity, thereby leaving sufficient active DHFR that is not inactivated by the antifolate anticancer drug (e.g. MTX) would result in antifolate drug resistance.

Reduced cellular retention of MTX polyglutamates can result from decreased FPGS activity due to decreased FPGS expression, and/or due to inactivating mutations in FPGS both of which lead to antifolate drug resistance.

Reduced cellular retention of polyglutamylatable antifolates (e.g., MTX) due to decreased FPGS activity resulting from either decreased FPGS expression or catalytically altered FPGS would result in antifolate drug resistance.

Increased FPGH activity due to enzyme overexpression would result in increased hydrolysis of antifolate drug polyglutamates and thereby lead to decreased antifolate anticancer drug retention as the various MRP1-3 will efficiently extrude the monoglutamate form of the antifolate (e.g., MTX).

Increased activity of the folate-dependent enzymes GARTF and AICARTF due to overexpression would assure that sufficient enzyme activity will be left [i.e., that activity which is not inactivated by the antifolate anticancer drug (e.g. MTA, AG2034 or DDATHF)], thereby allowing for sufficient de novo purine biosynthesis to occur.

Similarly, increased activity of TS due to overexpression, would leave sufficient enzyme activity [that is not bound to the antifolate drug (e.g., Tomudex, GW 1843 and MTA)] to synthesize uridylate and would therefore result in antifolate anticancer drug resistance.

Mutations in TS which disrupt antifolate binding but in the same time retain sufficient substrate (i.e., 5, 10-methylenetetrahydrofolate) binding and catalytic activity (thymidylate biosynthesis) would similarly result in antifolate anticancer drug resistance.

Increased activity of the multidrug resistance (MDR) efflux transporters Pgp and MRP1-3 due to transporter overexpression would lead to increased ATP-driven efflux of lipid-soluble antifolates such as trimetraxate and piritrexim that are Pgp substrates. In contrast, MRP overexpression will lead to increased efflux of hydrophilic antifolates in the monoglutamate form (e.g. MTX), resulting in both cases in antifolate drug resistance.

Thus, according to one aspect of the present invention a method is provided for the assessment of the responsiveness of tumor cells or a cancer patient to antifolate-containing chemotherapy. The method according to this aspect of the present invention is effected by searching for a mutation or mutations in a gene associated with folate uptake or metabolism in the tumor cells or in cells derived from the patient.

As used herein in the specification and in the accompanying claims, the term “responsiveness” refers to either an initial response of the patient or of tumor cells to antifolate chemotherapy or treatment, i.e., the response thereof to the first chemotherapy treatment, prior to which no exposure to antifolate compounds has been experienced; or a secondary response of the patient or of the tumor cells to antifolate-containing chemotherapy or treatment, i.e., the response to subsequent chemotherapy treatments, prior to which exposure to antifolate compounds has already been experienced.

As used herein in the specification and in the accompanying claims, the term “reduced folate” refers to compounds which are normally taken into cells by RFC, such as, but not limited to 5-methyltetrahydrofolate and 5-formyltetrahydrofolate (leucovorin).

As used herein in the specification and in the accompanying claims, the term “antifolate” refers to compounds which interfere with folate metabolism and are normally taken up into cells by RFC, such as, but not limited to, methotrexate, tomudex (raltitrexed, ZD1694), ZD9331, 5,10-dideazatetrahydrofolic acid (DDATHF), LY231514 (MTA), edatrexate and AG2034.

Candidate human genes which have been cloned and which participate in either folate uptake or metabolism include, but are not limited to, the human reduced folate carrier (RFC) gene (GeneBank Accession No. U19720, human dihydrofolate reductase (DHFR) gene (GeneBank Accession No. AF 201303.1), human folylpoly-γ-glutamate synthetase (FPGS) gene (GeneBank Accession No. NM 004957), human folylpoly γ-glutamate hydrolase (FPGH) gene (GeneBank Accession No. NM 003878), human glycinamide ribonucleotide transformylase (GARTF) gene (GeneBank Accession No. AF 008655.1), human phosphoribosylamino-imidazole-carboxamide transformylase (AICARTF) gene (GeneBank Accession No. D89976), human thymidylate synthase (TS) gene (GeneBank Accession No. AW 300821), human MDR1 gene (GeneBank Accession No. X58723), human multidrug resistance protein 1 (MRP1) gene (GeneBank Accession No. L05628), human multidrug resistance protein 2 (MRP2) gene (Individual GeneBank Accession Nos. for 12 individual exons) and human multidrug resistance protein 3 (MRP3) gene (GeneBank Accession No. AF 85690).

Searching for mutations in these genes of patients can be effected in tumor cells derived therefrom. Alternatively or additionally, searching To for mutations in these genes of patients can be effected in non-tumor cells derived therefrom. It will be appreciated in this respect that a mutation found in non-tumor cells of the patient is likely to also be present in tumor cells thereof.

The method according to this aspect of the present invention is further effected by evaluating an effect of mutation or mutations identified in the human gene or genes, on antifolate and reduced folate uptake in tumor cells derived from a cancer patient. Such evaluation can be effected by previous knowledge regarding the mutation or mutations detected. Alternatively, in the case of RFC, for example, such evaluation can be effected by evaluating the effect on uptake of the antifolate and the reduced folate by cells expressing a mutated RFC protein encoded by a polynucleotide harboring the mutation or mutations which were detected.

Various methods are known in the art which may be used to detect and characterize specific nucleic acid sequences and sequence changes. Nonetheless, as nucleic acid sequence data of the human genome, as well as the genomes of pathogenic organisms accumulates, the demand for fast, reliable, cost-effective and user-friendly tests for specific sequences continues to grow. Importantly, these tests must be able to create a detectable signal from a very low copy number of the sequence of interest. The following discussion examines three levels of nucleic acid detection currently in use: (i) signal amplification technology for detection of rare sequences; (ii) direct detection technology for detection of higher copy number sequences; and (iii) detection of unknown sequence changes for rapid screening of sequence changes anywhere within a defined DNA fragment. Each one of these approaches can be used to implement the method of the present invention.

Signal Amplification Technology Methods for Amplification:

The “Polymerase Chain Reaction” (PCR) technique comprises the first generation of methods for nucleic acid amplification. However, several other methods have been developed that employ the same basis of specificity, but create signal by different amplification mechanisms. These methods include the “Ligase Chain Reaction” (LCR), “Self-Sustained Synthetic Reaction” (SSR/NASBA), and “Qβ-Replicase” (Qβ).

Polymerase Chain Reaction (PCR): The polymerase chain reaction (PCR), as described in U.S. Pat. Nos. 4,683,195 and 4,683,202 to Mullis and Mullis et al., describe a method for increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polmerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method for amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes. Segev, PCT Publication No.

W09001069 A1 (1990). However, because the four oligonucleotides used in this assay can pair to form two short ligateable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) (Guatelli et al., Proc. Natl. Acad. Sci., 87:1874-1878, 1990), with an erratum at Proc. Natl. Acad. Sci., 87:7797, 1990) is a transcription-based in vitro amplification system (Kwok et al., Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo-and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta [symbol] Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55° C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method for the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999, 1990)

A similar 3′-mismatch strategy is used with greater effect to prevent ligation in the LCR (Barany, PCR Meth. Applic., 1:5, 1991). Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

Direct Detection Technology:

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including, Northern and Southern band RNase protection assays usually require the use of radioactivity and are not amenable to automation. Recently devised techniques have sought to eliminate the use of radioactivity and/or improve the sensitivity in automateable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), described by Urdea et al., Gene 61:253-264 (1987), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

Detection of Unknown Sequence Changes:

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet unknown mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucteotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perlman and Butow, Science 246:1106, 1989), but again, these are few in number.

Allele specific oligonucleotide (ASO): If the change is not in a recognition sequence, then allele-specific oligonucleotides (ASOs), can be designed to hybridize in proximity to the unknown nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation of an unknown character and position within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of unknown mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of unknown mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for the mutation or mutations in any of the genes listed above, such as, for example, the reduced folate carrier (RFC) gene, in tumor cells or in cells derived from a cancer patient is effected by a single strand conformational polymorphism (SSCP) technique, such as cDNA-SSCP or genomic DNA-SSCP. However, alternative methods can be employed, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

As shown in FIG. 12, according to another aspect of the present invention there is provided a kit for assessing a responsiveness of a cancer patient to antifolate chemotherapy, which is referred to hereinbelow as kit 20. Kit 20 includes a holder 22 which serves for holding at least one container 24 containing oligonucleotides, in a mixture or in individual packaging, in solution or dry form, capable of amplifying at least one fragment of a nucleic acid, either DNA or RNA, respresenting any of the genes listed above. Other containers 24 may include a DNA polymerase enzyme, such as a thermostable DNA polymerase, a reverse transcriptase enzyme, a mixture of dNTPs, a concentrated polymerase chain reaction buffer and a concentrated reverse transcription buffer. The oligonucleotides of kit 20 are preferably, but not limited to, those identified by SEQ ID NOs:1-20 (Table 3). Such nucleotides can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. Kit 20 according to preferred embodiments of the present invention further includes at least one precast gel 26 for executing single strand conformational polymorphism. Kit 20 may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. Kit 20 preferably includes a notice associated therewith in a form prescribed by a governmental agency regulating the manufacture, use or sale of diagnostic kits. Detailed instructions for use, storage and trouble shooting may also be provided with kit 20.

One ordinarily skilled in the art would be able to synthesize appropriate oligonucleotides which will enable amplification of sequences derived from any of the genes listed above. Thus, The nucleic acid sequences used while implementing the present invention may be amplified by known nucleic acid amplification protocols, such as, but not limited to, the polymerase chain reaction (PCR). To this end, there is provided a pair of oligonucleotides each of at least 17, preferably at least 18, more preferably between 19 and 25, still preferably, at least 26, say, 27-50 bases in length, which are specifically hybridizable with the nucleic acids described herein in an opposite orientation, so as to direct exponential amplification of a portion thereof in a nucleic acid amplification reaction, thereby obtaining a nucleic acid amplification product. The melting temperature (Tm) of the oligonucleotides of the pair of nucleotides is preferably selected similar, so as to enable hybridization thereof to target sequences under similar conditions. Such melting temperature can be estimated in advance using an appropriate software, such as, but not limited to the OLIGO software.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND EXPERIMENTAL METHODS

Materials:

[3′, 5′, 7′-³H]MTX (23.4 Ci/mmol) and [3′, 5′, 7′, 9′-³H]folic acid (25.5 Ci/mmol) obtained from Moravek Biochemicals (Brea, Calif.) were purified prior to use by thin layer chromatography (Jansen et al., 1989) and kept at −80° C. MTX was from Teva Pharmaceuticals Ltd., Israel, whereas folic acid, (d,1) leucovorin (calcium salt), and N-hydroxysuccinimide (NHS) were purchased from Sigma. G-418 (700-750 μg of active drug/mg) was obtained from GIBCO. The non-radioactive antifolate drugs were generous gifts from the following sources: GW1843U89, Dr. G. K. Smith (Glaxo Welcome); ZD1694 (Jackman and Calvert, 1995), Dr. A. Jackman (Institute for Cancer Research, Sutton, UK); PT523 (Rhee et al., 1994), Dr. W. T. McCulloch (Sparta Pharmaceuticals); DDATHF (Beardsley et al., 1989) and LY231514 (MTA) (Shih et al., 1997) (Lilly); AG2034 (Boritzki et al., 1996), Dr. T. J. Boritzki (Agouron Pharmaceuticals); Trimetrexate (Lin and Bertino, 1991), Dr. D. Fry (Parke-Davis).

Cell Cultures and GW1843U89 Selection:

Human CCRF-CEM leukemia cells, their MTX transport defective CEM/MTX subline (Rosoxvsky et al., 1980), as well as human breast cancer ZR-75-1 cells and their MTX transport-impaired MTX^{R}-ZR-75 cells, were grown in RPMI 1640 medium (containing 2.3 μM folic acid; Biological Industries, Beth Haemek, Israel) supplemented with 10% fetal calf serum (GIBCO), 2 mM glutamine, 100 U/ml penicillin G (Sigma), and 100 μg/ml streptomycin sulfate (Sigma). The growth medium of CEM/MTX cells contained 1 μM MTX (Rosowsky et al., 1980). The GW70 subline was established by stepwise exposure of parental CEM cells to gradually increasing concentrations of the potent thymidylate synthase inhibitor, GW1843U89 in normal growth medium; this selection was initiated at 1.5 nM and terminated at 70 nM GW1843U89, the cells were therefore termed GW70. GW70/LF cells were established by gradual deprivation of folic acid from the growth medium; this was terminated at a concentration of 5 nM folic acid.

Growth Assays:

In order to determine the leucovorin and folic acid growth requirement, as well as the MTX sensitivity, human leukemia CEM and GW70 cells and their clonal transfectants in transport defective mouse L1210/MTX^{r}A line (Schuetz et al., 1988; Schuetz et al., 1989; Brigle et al., 1995), exponentially growing cells in folic acid-free growth medium supplemented with 10% dialyzed fetal calf serum were first grown for 1-2 weeks in folate-free RPMI-1640 medium supplemented with 10% dialyzed fetal calf serum (Biological Industries, Beth Haemek, Israel) containing 200 μM glycine, 100 μM adenosine and 10 μM thymidine (GAT) and G-418 (750 pg active drug/ml, for transfectants). Thereafter, cells were washed twice with serum-free folic acid-free medium, in order to eliminate GAT. Cells were then seeded into 96-well plates (1×10⁵-2×10⁵ cells/ml; 0.15 ml/well) at the following densities: L1210, L1210/MTX^{r}A, CEM, and GW70 at: 1.5×10⁴, 2.3×10⁴, 3×10⁴, and 3×10⁴ cells/well, respectively. Thereafter, cells were exposed continuously to various concentrations of leucovorin or folic acid for 72 hours, following which cell numbers were determined by hemocytometer count of viable cells, using trypan blue exclusion. In the case of MTX, growth inhibition assays were performed in folic acid containing medium, supplemented with 10% fetal calf serum. The 50% inhibitory concentration (IC₅₀) is defined as the drug dose at which cell growth was inhibited by 50% as compared to untreated controls. EC₅₀, is defined as the folate cofactor concentration necessary to produce 50% of maximal cell growth.

Southern and Northern Blot Analyses:

High molecular weight genomic DNA was extracted from parental CEM cells and its various sublines. DNA was digested with EcoRI, fractionated by electrophoresis on 0.8% agarose gels, transferred to a Zetaprobe (Bio-Rad) nylon membrane, and UV crosslinked. RFC gene copy number was determined by Southern blot analysis using a [³²P]labeled (Feinberg and Vogelstein, 1983) human RFC cDNA probe isolated from a cDNA library derived from CEM-7A cells. Poly (A)⁺RNA was size-fractionated on 1% agarose/formaldehyde gels, blotted onto a GeneScreen⁺(New England Nuclear) nylon membrane and UV crosslinked. Human RFC cDNA and β-actin cDNA (Ponte et al., 1984) were labeled by random hexamer priming (Feinberg and Vogelstein, 1983). Southern and Northern blot hybridizations and post-hybridization washes were carried out under high stringency conditions, as previously described (Jansen et al., 1998). Southern and Northern blots were quantified with a BAS 1000 Bio-Imaging Analyzer (Fujix).

Western Blot Analysis:

To quantitate the levels of RFC expression, plasma membrane vesicles were isolated in the presence of 7 protease inhibitors as previously described (Freisheim et al., 1988). Plasma membrane proteins were resolved by electrophoresis on 10% polyacrylamide gels containing SDS and electroblotted onto a PVDF nylon membrane (Millipore). The blots were then blocked overnight at 4° C. in TBST buffer (150 mM NaCl/0.5% TWEEN 20/10 mM Fris-Cl at pH 8.0) containing 5% dry milk. The blots were then reacted with a polyclonal antiserum (1:1300; kindly provided by Dr. L. H. Matherly, Kannanos Cancer Institute, Detroit, Mich.) anti-recombinant human RFC for 3 hours at room temperature in TBST buffer, rinsed briefly with TBST, followed by three washes for 15 minutes in the same buffer. The blots were then reacted with horse-radish peroxidase-linked anti-rabbit antiserum (1:2500; Amersham) in TBST for 1 hour at room temperature, after which enhanced chemiluminescence detection was performed according to the manufacturers' instructions (Amersham).

Genomic PCR-SSCP Assay:

Genomic PCR-Single Strand Conformational Polymorphism (SSCP) analysis of the six exons of the human RFC gene was undertaken as a High-Throughput approach, in order to screen for mutations in tumor cells. In order to PCR the entire RFC coding region, 10 oligonucleotide primer pairs were designed, which span the entire RFC coding region, as detailed in Table 3.

TABLE 3
Oligonucleotide primer pairs targeting the entire RFC
coding region, used for genomic PCR

| | Upstream | | | | | | |
|---|---|---|---|---|---|---|---|
| | Primer (SEQ | | | Downstream | | | cDNA |
| Exon # | ID NO.) | | Sequence | Primer | | Sequence | position |
| 2 | Ex2Up | (1) | 5′ggatccttccagg | Ex2Dn | (2) | 5′ggtacccacatgc | 53-283^{a} |
| | | cacagtgtcac | | ctgctcccgcgtg | | | |
| 3 | Ex3Up | (3) | 5′gcggccgcacgc | RFC-2 | (4) | 5′gtaggaggaatag | 284-511 |
| | | ccgcctgtccgca | | gcgatgcgcgc | | | |
| | | gg | | | | | |
| 3 | RFC460 | (5) | 5′ggagctcttctaca | RFC750 | (6) | 5′cgcaccgccccc | 460-755 |
| | | gcgtcaccatggc | | ggtcgtcgc | | | |
| 3 | RFC680 | (7) | 5′ggtcctcgccctct | RFC950 | (8) | 5′cgtagtagaccac | 685-956 |
| | | tcctgaagcg | | caggtagtagcc | | | |
| 3 | RFC780 | (9) | 5′gcgcatgaatcct | Ex3Dn | (10) | 5′cgagggaatggc | 778-1043 |
| | | ggcccaggcg | | gtacccagcagcg | | | |
| 4 | Ex4Up | (11) | 5′tctcaggcgccat | Ex4Dn | (12) | 5′actcacgtggcga | 1044-1245 |
| | | cacgtccttcg | | tgggcacgagg | | | |
| 5 | Ex5Up | (13) | 5′ttgccgctttcagat | Ex5Dn | (14) | 5′tgaacccttgcgg | 1246-1387 |
| | | tgcatcttc | | accgggagg | | | |
| 6 | Ex6Up | (15) | 5′cctcacccggcttc | RFC1600 | (16) | 5′ggaaagcggcgg | 1388-1603 |
| | | tctttccag | | gctctgggctgg | | | |
| 6 | RFC1550 | (17) | 5′ggacaagggcctc | RFC1800 | (18) | 5′ggacagccagctg | 1556-1799 |
| | | ggaggcctgc | | gggacaagtc | | | |
| 6 | RFC1640 | (19) | 5′gcagagacagag | RFC1900 | (20) | 5′gcaaagttaccac | 1645-1898 |
| | | cgacccatacc | | aggggcgcc | | | |

^{a}Nucleotide numbering was according to Prasad et. Al. (1995), accession number U15939

Purified genomic DNA samples (0.5 μg) were amplified with Taq DNA polymerase (Promega) in a reaction buffer (50 μl) containing 10 pmoles of each primer, 0.2 mM unlabeled dNTP's and 10 μCi of [³²P]dATP in 1× reaction buffer (Promega) also containing 1.5 mM MgCl₂ and 5% DMSO. Samples were then heated to 95° C. for 5 min, 67° C. for 2 min, 72° C. for 3 min and amplified for 30 cycles of 94° C. for 1 min 65° C. for 2 min, 72° C. for 3 min followed by a 10 min extension at 72° C. The reaction was terminated by a 1:3 dilution in a stop solution consisting of 95% formamide, 20 mM EDTA, 0.05% bromophenol blue and 0.05% xylene cyanol. The amplified products, of about 150-300 bp, were denatured at 95° C. for 5 min, chilled on ice for 5 min and processed as follows: For exon 2, loading was either on 10% polyacrylamide gel containing 10% glycerol or 12.5% polyacrylamide gel lacking glycerol ran at 6 W at room temperature or at 4° C., respectively. The products of exon 2 were purified prior to electrophoresis with High Pure PCR purification kit (Boehringer Mannheim). For exon 3 cDNA nucleotide position 284-511 (Table 3-), loading was onto 10% polyacrylamide gel ran at 150 V at 22° C. overnight (˜16 hours). For the rest of exon 3, as well as for exons 4-6 (Table 3), loading was on 10% polyacrylamide gel ran at 17° C., 400 V for 3 hours. The gels were then dried and exposed to X-ray films. In addition, pre-casted 10% polyacrylamide gels were also used (ExcelGel DNA Analysis kit, Pharmacia) and ran at 700 V for 1.5 hour, using the Multiphor II Flatbed electrophoresis system, according to manufacturer's instructions. In this case, no radioactive nucleotide was used, and the PCR products were identified by silver staining.

Site Directed Mutagenesis and RFC cDNA Transfections:

An RFC cDNA clone containing the entire coding region (hRFC 1) was directionally cloned into pCDNA3(+) at the BamHI-XhoI site (Invitrogen). The various point mutations and the single nucleotide polymorphic variations were inserted in the native RFC cDNA by site directed mutagenesis (QuickChange, Stratagene). Mouse L1210/MTX^{r}A leukemia cells, which lack functional RFC transport activity (Schuetz et al., 1988; Schuetz et al., 1989; Brigle et al., 1995), were electroporated (300 V, 250-330 microfarads) with 40 μg of nonlinearized pCDNA3(+) harboring the native or truncated RFC cDNAs in serum-free RPMI-1640 medium and a final volume of 800 μl. Immediately thereafter, cells were diluted in prewarmed serum-containing growth medium (10 ml), allowed to recover for 32 hours, adjusted to 10⁵-2×10⁵ cells/ml in medium containing G418 (750 μg of active drug/ml), and distributed into 96-well plates at approximately 2×10⁴-4×10⁴ cells/well. After 10-20 days of incubation at 37° C., individual G418-resistant clones were verified microscopically, picked and expanded for further studies.

Transport Studies:

(Anti)folate transport measurements were performed essentially as previously described (Jansen et al., 1998). Briefly, cells were harvested in the mid-log phase, or collected from ALL patient donors, washed twice with Hepes-buffer saline solution (HBSS, pH 7.4) and concentrated to a density of 2×10⁷ cells/ml in an anion-free MHS buffer (1 ml) containing 20 mM HEPES, 225 mM sucrose at pH 7.4 (adjusted with MgO), 37° C. Uptake of [³H]MTX (specific activity 0.5 Ci/mmol) and [³H]folic acid (specific activity 1 Ci/mmol) was measured at an extracellular concentration of 2 μM in 1 ml of cell suspension at 37° C. For transport studies with [³H]folic acid, HBSS buffer was supplemented with 5 μM trimetrexate in order to block folic acid reduction (Assaraf and Goldman, 1997). At selected transport time intervals, ranging from 0.5-30 minutes, uptake was terminated by the addition of 10 ml ice-cold HBSS, after which centrifugation and cell wash with another 10 ml ice-cold HBSS were performed. The final cell pellet was then counted for radioactivity.

Patient Specimens:

Leukemia specimens, including B-precursor and T-cell ALL blasts, were obtained from newly diagnosed or relapsed patients with ALL, treated at the Children's Hospital of Michigan or the Free University Hospital, Amsterdam, the Netherlands. All patient samples were obtained after informed consent, and in accordance with protocols approved by the Committee on Investigation Involving Human Subjects at Wayne State University. Blast cells were separated from bone marrow by standard Ficoll Hypaque density centrifugation. Specimens were documented for patient age, sex, race, presenting white blood cell count, percent blast cells in bone marrow, and immunophenotype. B-precursor patients were treated with assorted treatment protocols, generally including MTX during consolidation (1 g/m²) and maintenance (20 mg/m²) phases. Chemotherapy for T-ALL was typically more intensive than for B-precursor ALL, and frequently included anthracyclines and epipodophyllotoxins, with or without intermediate dose intravenous MTX. Genomic DNA was isolates from 3-5×10⁶ blasts using the Puregene DNA isolation kit (Gentra Systems, Minneapolis, Minn.).

### EXPERIMENTAL RESULTS

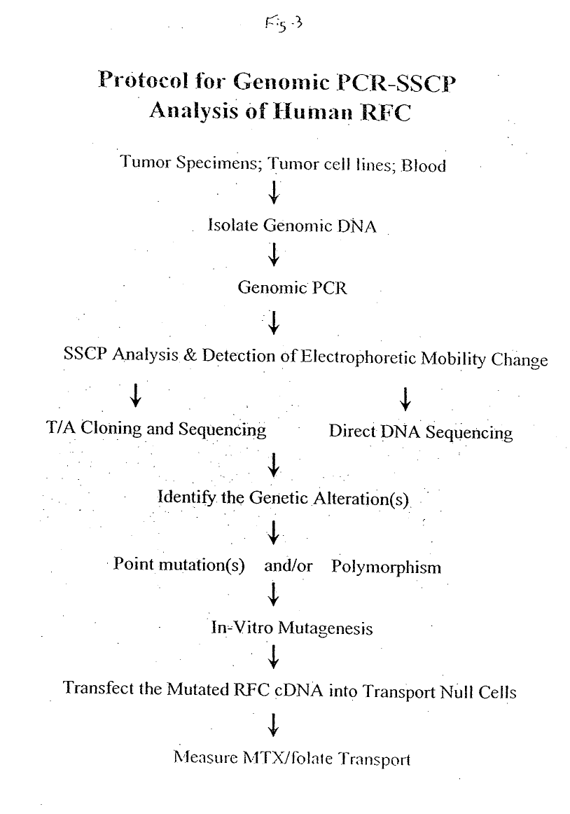

In order to screen for genetic alterations occurring in the RFC gene in tumor cells, a high throughput genomic PCR-SSCP assay has been developed, the steps of which are presented in FIG. 3. Initially, DNA is extracted from blood of healthy individuals, tumor cell lines or tumor specimens. Following genomic PCR with specific oligonucleotide primers, spanning the entire RFC coding region (Table 3), single strand DNA products are examined for aberrant mobility on native polyacrylamide gels, capable of detecting a single nucleotide change in a fragment as large as 300 nucleotides. These altered amplified DNA products are sequenced and the role of the observed mutation or polymorphic variation on (anti)folate transport, antifolate growth inhibition, as well as folate growth requirement, is determined by transfection of the mutant RFC cDNA into transport deficient cells.

It has been recently shown by the inventors of the present invention (Jansen et al., 1998) that the transport impairment of human leukemia CEM/MTX cells is due to a single G to A mutation at nucleotide 227 at exon 2 (using the numbering of Prasad et al., 1995), thus resulting in a lysine to glutamate substitution at amino acid residue 45, predicted to reside within TMD1 of RFC (Jansen et al., 1998 and Table 4).

TABLE 4
Summary of RFC mutations identified by the SSCP assay in
antifolate-resistant tumor cells and leukemia specimen

| Putative | Exon | Amino-acid | | Nucleotide(s) | Nucleotide | |
|---|---|---|---|---|---|---|
| Location | Number | Position | Change | Position | Change | Cells |
| TMD1 | 2 | 45 | Glu-Lys | 227^{a} | G-A | CEM/MTX |
| TMD1 | 2 | 44 | Gly-Arg | 224 | G-A | |
| | 4 | 393 | Frame | 1139-40 | Insertion of | |
| | | (termination) | Shift | | G | |
| TMD2 | 3 | 86 | Leu-Leu | 352 | C-T | |
| TMD1 | 2 | 29 | Val-Leu | 179 | G-C | GW70^{b} |
| TMD1 | 2 | 45 | Glu-Lys | 227 | G-A | |
| TMD1 | 2 | 46 | Ser-Ile | 331 | G-T | |
| TMD1 | 2 | 29 | Val-Leu | 179 | G-C | GW70/LF^{b} |
| TMD1 | 2 | 45 | Glu-Lys | 227 | G-A | |
| TMD1 | 2 | 46 | Ser-Ile | 331 | G-T | |
| L2 | 3 | 88 | Asp-His | 356 | G-C | |
| | 4 | 361 | Frame | 1061-65 | 5 bp | MTX^{R}-ZR-75 |
| | | (termination) | Shift | | deletion | |
| N-terminus | 2 | 27 | Arg-His | 174 | G-A | M1 (Patient)^{c} |
| L1 | 2 | 56 | Asp-His | 261 | G-C | |
| C-terminus | 6 | 522 | Asp-Asn | 1635 | G-A | J-7 (Patient) |

^{a}Nucleotide numbering was according to Prasad et al. (1995), accession number U15939.
^{b}The 3 mutations in GW70 and 4 mutations in GW70/LF reside on the same RFC allele.
^{c}The two genetic alterations identified in M1 Leukemia specimen reside on the same RC allele of which Arg27 His is attributable to polymorphic variation.

Furthermore, additional C to T mutations at nucleotides 352 and 791, both in exon 3, were also identified in these cells; however, these mutations were silent ones, thus retaining the native leucine at amino acid 86 and proline at amino acid 232 (Table 4). Hence, the ability of the genomic PCR-SSCP assay to detect these established mutations in DNA from CEM-MTX cells was examined first.

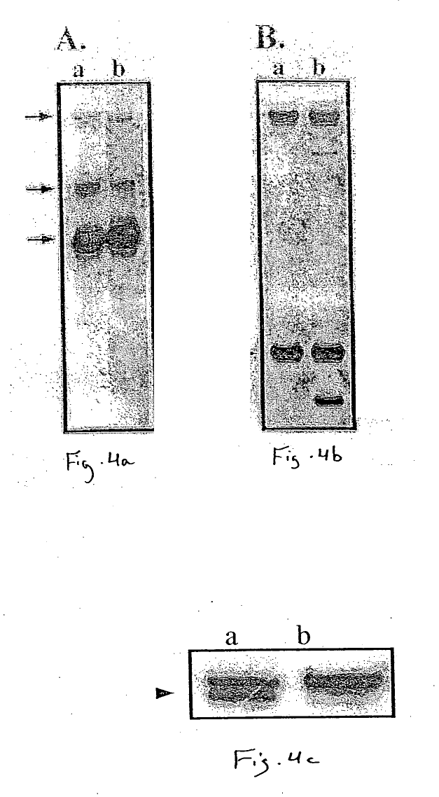

Utilization of the genomic PCR-SSCP assay to analyze CCRF-CEM cells has revealed that whereas parental cells contained three distinct bands in exon 2 (FIG. 4a, lane a, arrows) and two distinct bands in exon 3 (FIG. 4b lane a, arrows), an additional band in exon 2 (FIG. 4a, arrowhead) and two additional bands in exon 3 were present in DNA from CEM-MTX cells (FIG. 4b, lane b, arrowheads). Similar PCR-SSCP analysis of genomic DNA from MTX transport deficient sublines CEM-T revealed an additional band in exon 4 (FIG. 4c, lane a, arrowhead). These altered DNA products, with aberrant electrophoretic mobility, were sequenced and found to harbor the exact mutations hereinabove described (Table 4).

The mouse RFC gene was originally cloned by functional complementation (Dixon et al., 1994) of the severe transport defect in MTX-resistant MTX^{R}-ZR-75 human breast tumor cells (Cowan and Jolivet, 1984). However, the genetic lesion underlying this MTX transport defect has never been identified. Thus, DNA from MTX^{R}-ZR-75 and their parental ZR-75 cells, was also examined by the genomic PCR-SSCP assay, such that the entire RFC coding sequence contained in exons 2 through 6 was analyzed (FIG. 5). An intense band with an aberrant migration (FIG. 5, lane h, asterisk) was present in exon 4 of MTX^{R}—ZR-75 cells (FIG. 5, lane g vs. h). Sequence analysis of this altered amplified DNA product revealed a 5 bp deletion of nucleotides 1061-1065, resulting in frame-shifting and premature translation termination (Table 4-).

Table 4 summarizes RFC mutations identified by the SSCP assay in antifolate-resistant tumor cells and leukemia specimens. Nucleotide numbering is according to Prasad et al., 1995, GeneBank accession number U15939. The 3 mutations in GW70 and 4 mutations in GW70/LF reside on the same RFC allele. The two genetic alterations identified in M1 Leukemia specimen reside on the same RFC allele of which Arg27His is attributable to polymorphic variation, as is further detailed herein. FIG. 13 provides a graphic depiction of the location of the RFC missense mutations listed in Table 4.

As shown in Table 5 below, both GW70 and GW70/LF cells did not show any appreciable change in the activity levels of TS, FPGS and FPGH, as compared to parental CEM cells.

TABLE 5
Activities of TS, FPGS, FPGH and folate transport activity in CEM/WT
and their GW1843-resistant sublines^{a} Enzyme Activity

| | | | |
|---|---|---|---|
| Cell Line | TS^{b} | FPGS^{c} | FPGH^{d} |

| | | | | | | |
|---|---|---|---|---|---|---|
| CEM/WT | 3.6 | 0.8 | 1300 | 115 | 59 | 15 |
| GW70 | 2.5 | 0.7 | 1237 | 197 | 46 | 10 |
| GW70-LF | 2.6 | 1.5 | 983 | 38 | 61 | 11 |

^{a}Results are the means S.D. of 3-5 experiments;
^{b}TS activity (measured at 10 M dUMP) is expressed as nmol/h/mg protein;
^{c}FPGS activity (measured using 250 M MTX as a substrate) is given as pmol/h/mg protein;
^{d}FPGH activity (measured using 100 M MTX diglutamate as a substrate is given as nmol/h/mg protein;
^{e}Uptake of MTX was measured at 2 M [³H] MTX and expressed as pmole/min/10⁷ cells;
^{f}Uptake of folic acid was measured at 2 M [³H] folic acid and expressed as pmole/min/10⁷ cells.

Growth Inhibition by Antifolates and Folates Growth Requirement of CEM and GW70 Human Leukemia Cells:

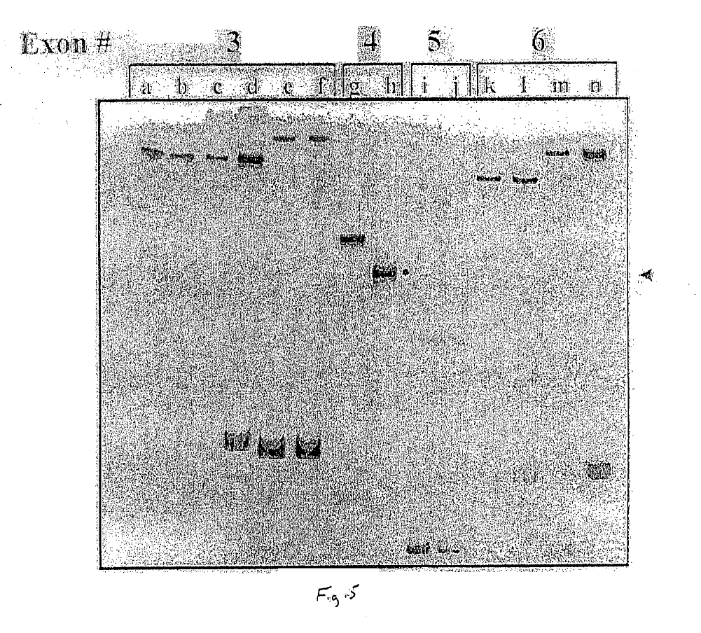

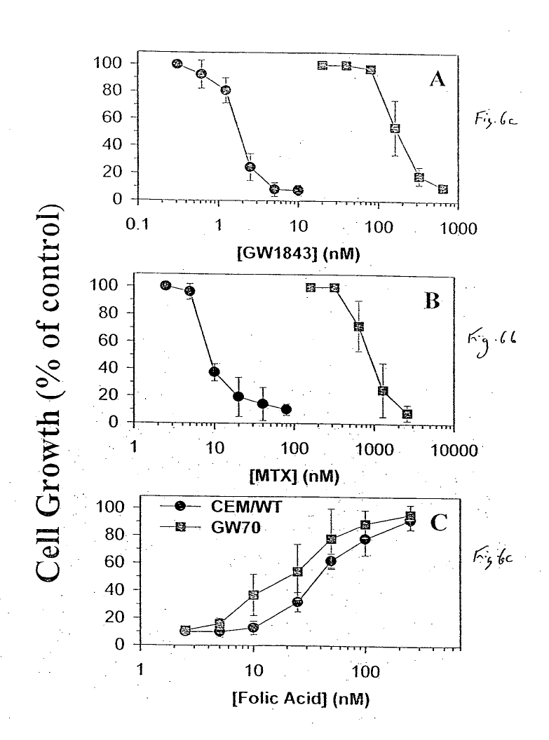

GW1843U89 is an extremely potent folate-based inhibitor of thymidylate synthase, with a novel methyl-oxo-benzoquinazoline structure (Hanlon and Ferone, 1996). Despite the bulky structure of GW1843U89, the human RFC displays the highest affinity for this compound as a transport substrate (Kᵢ=0.8 μM), even when compared to reduced folates (Westerhof et al., 1995). Thus, to explore whether structural alterations in the human RFC could impair GW1843U89 uptake and result in resistance to this novel antifolate, a human leukemia CEM-GW70 cell line was established by stepwise selection of CEM cells in gradually increasing GW1843U89 concentrations, as described in the experimental methods section above. GW70 cells did not show any change in the activities of thymidylate synthase, dihydrofolate reductase, folylpoly-γ-glutamate synthetase or folylpoly-γ-glutamate hydrolase. However, GW70 cells displayed a 100-fold resistance to GW1843U89 (FIG. 6a and Table 6) and cross-resistance to MTX (FIG. 6b and Table 6). Furthermore, GW70 cells also displayed a marked cross-resistance (15-147-fold) to various antifolates, that depend on RFC for their cellular uptake, including ZD1694, ZD9331, LY231514 (MTA), DDATHF, and PT523, but were collaterally more sensitive than parental CEM cells, to the lipid-soluble antifolates trimetrexate and AG337. Despite this RFC-dependent high level resistance to antifolates, GW70 cells displaced a 2-fold decrease in the folic acid growth requirement (FIG. 6c and Table 6), and had a relatively small increase in the leucovorin growth requirement (Table 6). Altogether, these results were suggestive of an altered RFC-mediated transport, as is further described herein.

Table 6 below summarizes the antifolate growth inhibition and folate growth requirement in parental human CEM leukemia cells and their GW70 subline. Antifolate activity is presented as IC₅₀, the antifolate concentration (GW1843 and MTX, nM) resulting in 50% cell growth inhibition, after 72 hours drug exposure. Folate growth requirement is presented as EC₅₀, folate concentration (folic acid and leucovorin, nM) resulting in 50% of maximal control cell growth.

TABLE 6
Antifolate growth inhibition and folate growth requirement
in parental human CEM leukemia cells and their GW7Q subline

Cell line

| Anti(folate) | CEM/WT | GW |
|---|---|---|
| GW1843 | 1.8 ± 0.22^{a} | 171.7 ± 34.5 |
| MTX | 8.7 ± 0.6^{a} | 888.7 ± 34.5 |
| Folic Acid | 44.9 ± 3.7^{b} | 24.2 ± 7.1 |
| Leucovorin | 0.51 ± 0.08^{b} | 2.4 ± 1.1 |

^{a}IC50, antifolate concentration (nM) that inhibits cell growth by 50% following 72 h drug exposure. Results are the mean ± S.D. of 4-6 independent experiments.
^{b}EC50, folic acid or leucovorin concentration (nM) necessary to support 50% of maximal growth of control cultures.

Table 7 below summarizes the kinetic parameters of folic acid and MTX transport in CEM cells and the GW1843-resistant sublines.

TABLE 7
Kinetic parameters of MTX and folic acid transport in parental
CEM cells and their GW 1843 - resistant sublines^{a}

| | MTX | | Folic Acid | |
|---|---|---|---|---|
| | | Vmax | | Vmax |
| | Km | (pmol/ | Km | (pmol/ |
| Cell line | (μM) | min/10⁷) | (μM) | min/10⁷) |
| CEM/WT | 5.1 ± 1^{b} | 4 ± 0.7 | 175 ± 39 | 3.6 ± 1.5 |
| CEM/GW70 | 16 ± 3.9 | 1.6 ± 0.7 | 16.5 ± 3.4 | 4.2 ± 1.7 |
| CEM/GW70-LF | 17.1 ± 1.2 | 82.9 ± 9.2 | 15.5 ± 2.5 | 52 ± 5.2 |

^{a}The kinetic parameters Km and Vmax for [³H] MTX and [³H] folic acid influx were obtained from Lineweaver-Burk plots of initial (3 min) uptakes rates performed at extracellular (anti)folate concentrations of 1-200 M;
^{b}Results are the mean S.D. of four separate experiments performed in duplicates.

Transport Studies, Kinetic Transport Parameters and Affinities for Folic Acid and MTX in CEM, GW70 and GW70/LF Cells:

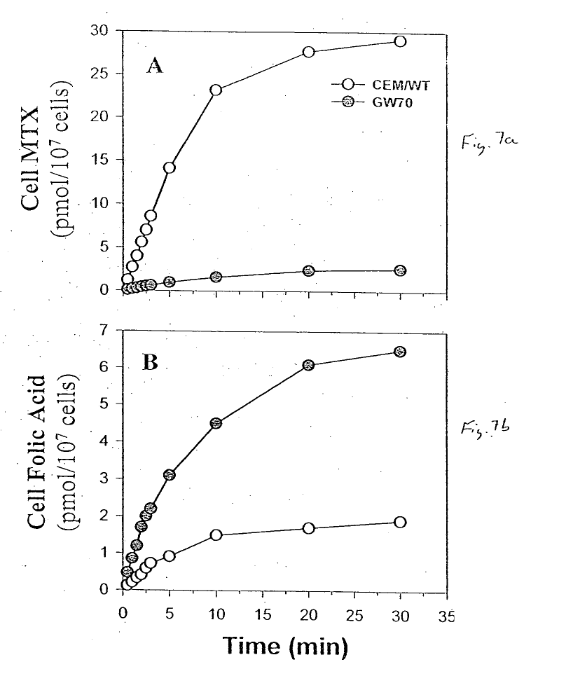

Transport analysis was conducted by monitoring radioactive MTX or folic acid uptake into cells in suspension. [³H]MTX influx in GW70, is as compared to CEM cells, was decreased >10-fold (0.3±0.1 vs. 3.2±0.7 pmol/min/10⁷ cells; n=12); this was accompanied by a consistent reduction in the steady-state MTX levels (FIG. 7a). These changes were associated with a 3-fold in the transport Km for MTX and a 2.5-fold decrease in the transport Vₘₐₓ (Table 7). In contrast, GW70 cells displayed a 10-fold decrease in the transport Kₘ for folic acid, while retaining the parental Vₘₐₓ for this folate (Table 7). Consequently, and consistent with the 2-fold decrease in the folic acid growth requirement, as is described hereinabove (FIG. 6c and Table 6), GW70, as is compared to their parental CEM cells, exhibited a >3-fold increase in both the influx (0.19±0.05 vs. 0.66±0.17 pmol/min/10⁷ cells, respectively; n=8) and steady-state transport levels of folic acid, under conditions, in which folic acid reduction was blocked by trimetrexate (FIG. 7b).

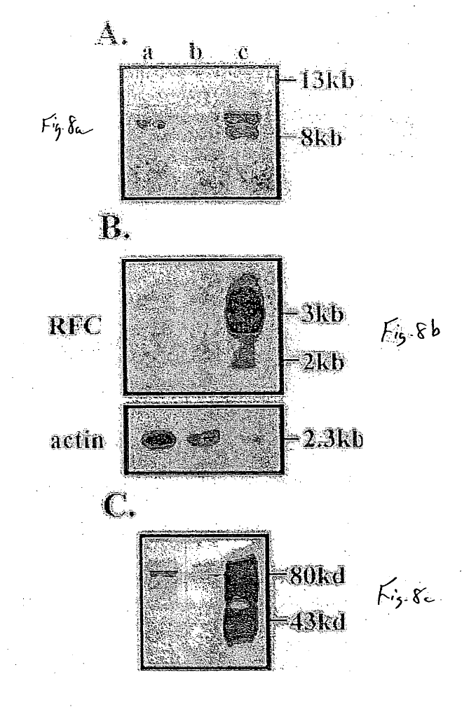

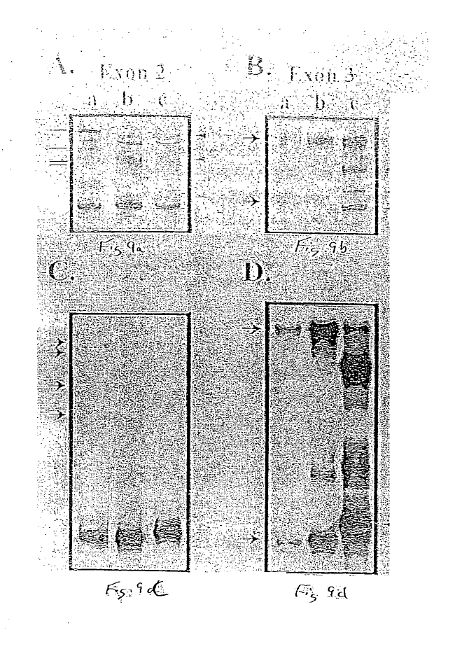

The impaired antifolate transport via RFC in GW70 made transport kinetic measurements difficult. To promote RFC overexpression as recently shown (Jansen et al., 1998), GW70 cells were gradually adapted to grow on low folic acid concentrations, resulting in the establishment of GW70/LF cells, which required only sub-nanomolar leucovorin concentrations for their growth. Indeed, GW70/LF cells to displayed 5-fold RFC gene amplification (FIG. 8a), consequent overexpression of the 3.1 kb (native) and 2.1 kb (truncated) RFC mRNAs (FIG. 8b), thus resulting in a prominent overproduction of the native ˜80 kDa and truncated 43 kDa RFC proteins (FIG. 8c). This was associated with a 21-fold and 15-fold increase in the transport Vₘₐₓ for MTX and folic acid, respectively, in GW70/LF cells as compared to their parental cells (Table 7). This carrier overexpression in GW70/LF cells facilitated the detection of a marked increase in the transport affinity not only for folic acid (Table 7-) but also for leucovorin and 5-methyltetrahydrofolate and for DDATHF (Table 8-). In contrast, the transport affinity for GW1843U89 and ZD1694 was notably decreased in GW70/LF cells (Table 8-).

Table 8 below summarizes the affinities, presented as Kᵢ^{app} (μM) (anti)folate concentration eliciting 50% inhibition of [³H]MTX influx, the latter presented at an extracellular concentration of 5 μM) of RFC from CEM-7A cells and antifolate-resistant cells, for various folate cofactors and folate analogs.

TABLE 8
Affinities of RFC from CEM-7A and antifolate-resistant
cells for various folate cofactors and folate analogs^{a}

| | | | Kᵢ app (M) | | | |
|---|---|---|---|---|---|---|
| (Anti)folate | CEM/GW70-LF | | CEM/MTX-LF | | CEM-7A | |
| Folic Acid | 21.9 | 2.7^{b} | 12.3 | 3 | 550 | 95 |
| Leucovorin | 1.5 | 0.1 | 0.29 | 0.06 | 4.5 | 1.1 |
| 5-methyl-THF | 1.3 | | 0.5 | 0.11 | 4.2 | 0.9 |
| ZD1694 | 7.3 | 1 | 2.7 | 0.6 | 2 | 0.5 |
| GW1843U89 | 4.7 | 0.1 | 2.6 | 0.6 | 0.8 | 0.2 |
| PT523 | 2.4 | 0.6 | 0.65 | 0.23 | 1.2 | 0.2 |
| DDATHF | 2.4 | 0.5 | 0.42 | 0.04 | 5.5 | 1.4 |

^{a}The affinities of RFC for folate and antifolate substrates are given as the (anti) folate concentration (M) eliciting 50% inhibition of [³H] MTX influx where the latter was at an extracellular concentration of 5 M;
^{b}Results are the mean of S.D. of 3-4 separate experiments.

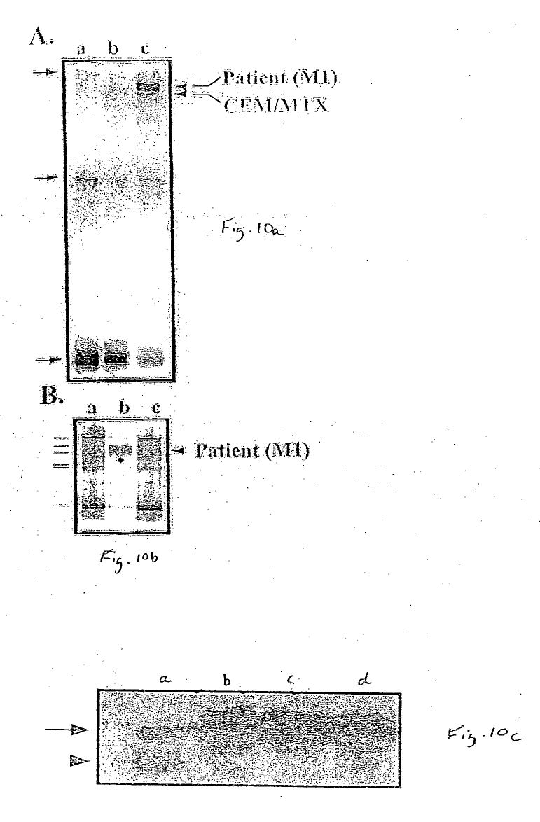

To characterize the RFC mutation(s) which may underlie this defective-transport based, antifolate resistance, DNA from GW70 and GW70/LF cells was subjected to the PCR-SSCP assay. GW70 (FIGS. 9a and 9c, lane b) and GW70/LF cells (FIGS. 9a and 9c, lane c) displayed an identical electrophoretic mobility pattern in exon 2, which was drastically different from that observed with parental CEM cells (FIGS. 9a and 9c, lane a). Sequence analysis revealed that both GW70 and GW70/LF cells contained in exon 2 three identical mutations, including a G to C, G to A, and G to T at nucleotide positions 179, 227, and 331, respectively (see Table 4, above). These mutations resulted in the following amino acid substitutions: Val 29 to Leu, Glu 45 to Lys, and Ser 46 to Ile, respectively (Table 4-, above). Additionally, whereas CEM (FIGS. 9b and 9d, lane a) and GW70 cells (FIGS. 9b and 9d, lane b) had a normal electrophoretic mobility pattern for exon 3 of RFC, GW70/LF cells (FIGS. 9b and 9d, lane c) displayed a markedly altered pattern. Sequencing identified a G to C mutation at nucleotide 356 in exon 3, resulting in a substitution of His 88 for Asp (Table 4-, above). Furthermore, whereas parental CEM cells were heterozygous, thus containing two RFC species, one with Arg 27 (G at nucleotide 174) and another with His 27 (A at nucleotide 174), GW70 cells lost this heterozygosity and therefore contained only the Arg 27 allele. This loss of heterozygosity was consistent with the genomic rearrangements observed in the Southern analysis with DNA from both GW70 and GW70/LF cells when compared to parental CEM cells (lanes b, c vs. lane a in FIG. 8a).

Table 9 below summarizes the intracellular folate pools in parental CEM cells and their GW-resistant sublines in parental CEM cells and their GW1843-resistant sublines.

TABLE 9
Intracellular reduced folate pools in parental CEM cells and their
antifolate-resistant sublines

| | CH₂THF + | | | | | | | | |
|---|---|---|---|---|---|---|---|---|---|
| Cell Line | THF | | 5-CH₃THF | | DHF | 10-CHO-THF | | TOTAL | |
| GEM/WT | 24 | 1^{a} | 9.9 | 3.1 | <0.3 | 37 | 2 | 70 | 3 |
| GW70 | 44 | 3 | 37 | 10 | <0.3 | 82 | 9 | 163 | 16 |

| | | | | | | | | | |
|---|---|---|---|---|---|---|---|---|---|
| GW70/LF^{b} | 3.4 | 0.7 | 0.4 | 0.3 | 3.9 | 0.9 | <0.3 | 7.7 | 0.4 |

| | | | | | | | | | |
|---|---|---|---|---|---|---|---|---|---|
| GW70/LF^{c} | 92 | 10 | 39 | 14 | <0.3 | 173 | 28 | 303 | 34 |

| | | | | | | | | |
|---|---|---|---|---|---|---|---|---|
| CEM/MTX^{d} | 13.9 | 0.5 | <0.3 | 6 | 0.8 | <0.3 | 21.0 | 1.1 |

^{a}Intracellular reduced folate levels (pmol/mg protein) values represent the means SEM from six separate determinations;
^{b}Cells grown In the presence of 2 nM folic acid;
^{c}Cells grown In the presence of 2.3 M folic acid;
^{d}Data presented are derived from Jansen et al., 1998.

Thus, as shown below in Table 10, GW70 cells displayed a high level resistance to various antifolate anticancer drugs which use RFC as the main uptake route. In contrast, GW70 cells were hypersensitive to lipid-soluble antifolates including trimetraxate which do not use the RFC for their entry.

TABLE 10
The growth inhibitory effects of antifolates on human CEM cells and
their GW1843-resistant sublines^{a}

| Cell Line | GW1843 | MTX | ZD1694 | MTA | ZD9331 | AG337 | DATHF | TMQ | PT523 |
|---|---|---|---|---|---|---|---|---|---|
| CEM/WT | 2.4 ± 0.4 | 8.1 ± 2.2 | 3.5 ± 0.6 | 23 ± 7 | 16 ± 1 | 1800 ± 340 | 11 ± 4 | 7.5 ± 2.1 | 1.1 ± 0.3 |
| GW70 | 123 ± 48 | 775 ± 81 | 515 ± 224 | 351 ± 73 | 479 ± 67 | 974 ± 241 | 360 ± 106 | 7.2 ± 1.7 | 81 ± 16 |
| GW70/LF | 2.5 ± 0.7 | 8.0 ± 0.1 | 3.2 ± 0.5 | 4.6 ± 0.6 | 25 ± 6 | 920 ± 430 | 8.2 ± 1.7 | 1.1 ± 0.2 | 1.4 ± 0.1 |
| GW70/LF^{b} | 7.5 ± 1.5 | 54 ± 11 | 389 ± 55 | 221 ± 47 | 12 ± 4 | 2220 ± 264 | >10000 | ND^{c} | ND |

^{a}Data presented are IC₅₀ (nM) values obtained after 72 h drug exposure;
^{b}Cells grown in the presence of 2.3 M folic acid;
^{c}Not determined

Genomic PCR-SSCP Assay Screening Tumor Specimens:

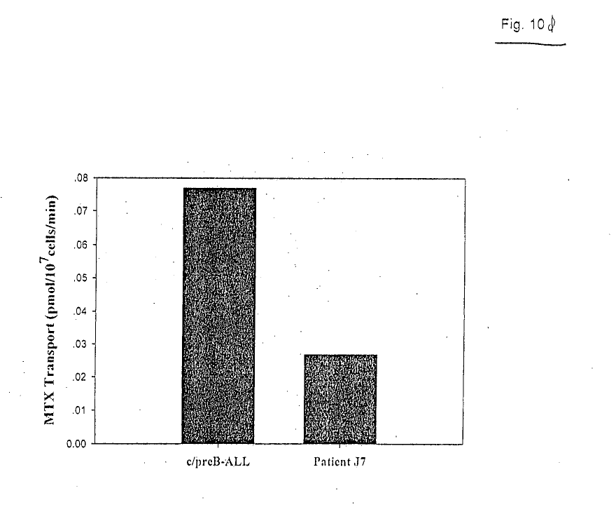

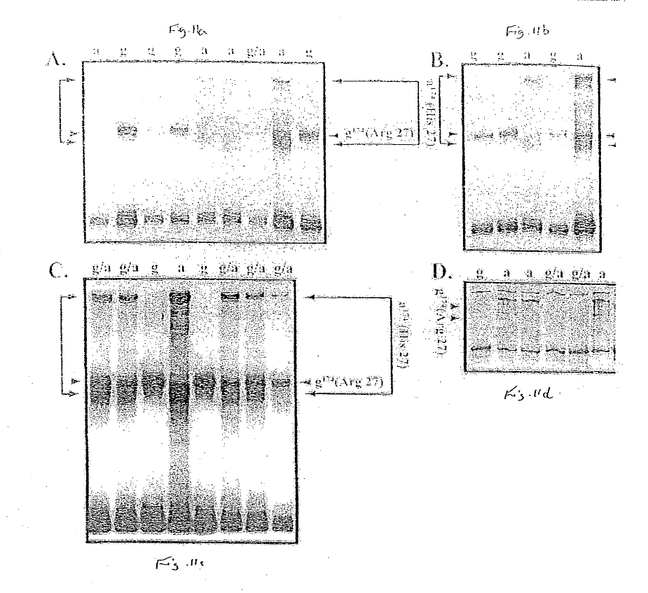

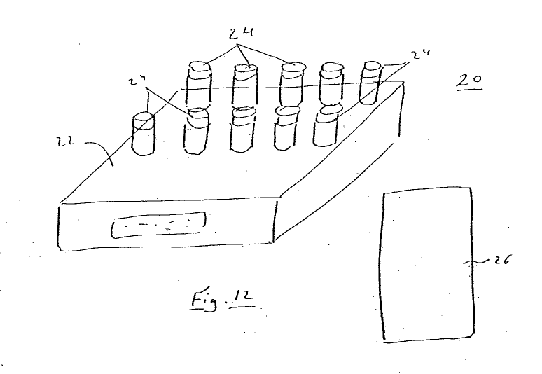

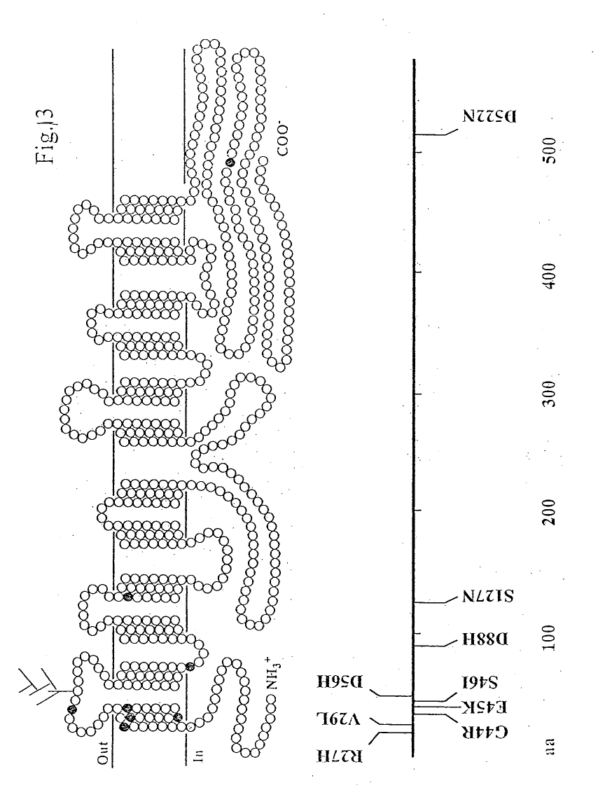

The applicability of the genomic PCR-SSCP assay for the screening of RFC mutations in tumor specimens was further explored. Thus, DNA from a total of 25 B-precursor and T-cell childhood acute lymphoblastic leukemia (ALL) specimens (the vast majority of which were derived at diagnosis) were screened. One DNA specimen obtained at diagnosis from a B-precursor ALL patient (termed M1) which relapsed 15 month later, displayed an altered electrophoretic mobility pattern in exon 2 (FIGS. 10a and 10b, lane b, marked also by asterisk). Sequencing revealed that this patient contained a G to C mutation at nucleotide 261, which resulted in a substitution of His 56 for Asp in the first predicted loop (L1) of RFC, and at the same time contained a G to A shift at nucleotide 174 in the same exon, and was thus homozygous for His 27 (Table 4, above). Interestingly, SSCP analysis of 30 DNA samples from both healthy individuals and leukemia patients identified three equally distributed polymorphic groups at nucleotide position 174: one third of the individuals were homozygous for G174, thus containing Arg 27 (FIGS. 11a-11d, arrowhead), the other third was homozygous for A174, thus displaying His 27 (FIGS. 11a-11d, arrows), and the remaining group was heterozygous, namely containing G and A at nucleotide 174, thus expressing two RFC species, one with Arg 27, and another with His 27 (FIGS. 11a-11d, arrowhead and arrows).

Another DNA specimen obtained at diagnosis from a B-precursor ALL patient (termed J7) displayed an altered electrophoretic mobility pattern for exon 6 (FIG. 10c, lane a). Sequencing revealed that this patient contained a G to A mutation at nucleotide 1635, which resulted in a substitution of Asn 522 for Asp in the C-terminus region of RFC (Table 4, above). Comparison of MTX uptake in blast cells from B-precursor ALL patient J7 and other leukemia patients having intact RFC (FIG. 10d) clearly demonstrates impaired antifolate transport resulting from the Asp522Asn mutation. As would be expected in view of the findings described herein, patient J7 was fatally chemotherapy resistant.

Transfection of Aberrant RFC cDNA into MTX-A Cells:

To study the role of the His 56 mutation identified in the ALL patient, as well as the polymorphic His 27 variation, on antifolate resistance and folate growth requirement, RFC cDNAs harboring either or both of these genetic alterations, were stably transfected into transport-deficient mouse leukemia MTX^{r}A cells. A dominant mutant was used as control: the Glu 45 Lys RFC mutation recently shown to play a major contributing role in the >200-fold MTX resistance, due to a severe impairment of MTX transport while preserving folic acid and leucovorin uptake (Jansen et al., 1998). Thus, a parameter was defined, calculated as the ratio of MTX growth inhibition EC₅₀ by the folate cofactor growth requirement IC₅₀; this ratio was a useful parameter, as the higher the ratio value is, the more MTX resistant the cells are, and the lower folate cofactor concentration is required. Transfection of the Glu 45 Lys mutation into transport null cells, resulted in markedly increased ratio of MTX IC₅₀/folic acid EC₅₀, as compared to the ratio obtained with native RFC (R27) transfection; these results demonstrated the prominent MTX resistance and decreased folic acid growth requirement, as described in Table 11 below.

TABLE 11
MTX growth inhibition and folate growth requirement in mouse
MTX^{r}A leukemia transfectants harboring either the native RFC
(Arg27) or altered RFC cDNA

| Glu45Lys | His27/His56 | Arg27/His56 | His27 | Arg27 | (Anti) folate |
|---|---|---|---|---|---|
| 15.3 ± 2.3 | 13.7 ± 1.4 | 8.7 ± 0.5 | 9.5 ± 2.5 | 7.9 ± 0.4 | MTX^{a} |
| (1.94) | (1.74) | (1.1) | (1.2) | (1.0) | |
| 0.65 ± 0.15 | 0.42 ± 0.1 | 0.7 ± 0.14 | 0.78 ± 0.09 | 0.76 ± 0.03 | Leucovorin^{b} |
| (1.17) | (1.81) | (1.32) | (0.97) | (1)^{c} | |
| 3.2 ± 0.8 | 5.1 ± 1.2 | 13.1 ± 2.3 | 14.6 ± 0.5 | 14.4 ± 1.7 | Folic acid^{b} |
| (4.5) | (2.82) | (1.1)) | (0.99) | (1)^{d} | |

^{a}IC₅₀, MTX concentration (nM) which inhibits cell growth by 50% following 72 h drug exposure.
^{b}EC₅₀, leucovorin or folic acid concentration (nM) necessary to support 50% of maximal growth of control cells. Results are the mean ± S.D. of 5-6 independent experiments.
^{c}MTX IC₅₀/leucovorin EC₅₀ ratio for each transfectant.
^{d}MTX IC₅₀/folic acid EC₅₀ ratio for each transfectant.

Transfection of either the polymorphic His 27 or the His 56 mutant RFC cDNAs alone, neither changed MTX sensitivity, nor the folate growth requirement, as reflected by the constant ratios of MTX IC₅₀/folate EC₅₀, as compared to native RFC (Arg27) transfection (Table -11). Interestingly however, transfection of the double His 27/His 56 mutant present in the ALL patient M1, rendered cells more resistant to MTX than transfectant cells expressing the native RFC (i.e., Arg 27) (Table 11). More importantly, this double mutant transfectant which exhibited MTX resistance, also displayed a notable decrease in both the folic acid and leucovorin growth requirements, relative to the native RFC cDNA transfectant; which is well reflected in the prominently increased ratio of MTX IC₅₀/folate EC₅₀ (Table 11). These surprising results indicate that this double His 27/His 56 mutant RFC confers MTX resistance, but at the same time equips the cells with an increased ability to accumulate folic acid and leucovorin (Table 11).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications cited herein, including sequences identified by their GeneBank Accession Nos., are incorporated by reference in their entirety. Citation or identification of any reference in this application shall not be construed as an admission that such reference is available as prior art to the present invention.

References:

1. Allegra, C. J., Chabner, B. A., Drake, J. C., Lutz, R., Rodbard, D., and Jolivet, J. (1985) J. Biol. Chem 260:9720-9726.

2. Allegra, C. J., Drake, J. C., Jolivet, J., and Chabner, B. A. (1985) Proc. Natl. Acad. Sci. USA. 82: 4881-4885.

3. Allegra, C. J., Hoang, K., Yeh. G. C., Drake, J. C., and Baram J. (1987) J. Biol. Chem. 262: 13520-13526.

4. Antony, A. C. (1992) Blood 79: 2807-2820.

5. Assaraf, Y. G., and Goldman, I. D. (1997) J. Biol. Chem. 272: 17460-17466.

6. Assaraf, Y. G., and Schimke, R. T. (1987) Proc. Natl. Acad. Sci. USA. 84: 7154-7158.

7. Assaraf, Y. G., Babani, S., and Goldman, I. D. (1998) J. Biol. Chem. 273: 8106-8111.

8. Baggott, J. E., Vaughn, Y. M., and Hudson, B. B. (1986) Biochem. J 236: 193-200.

9. Bannwarth, B., Labat, L., Moride, Y., and Schaeverbeke, T. (1994) Drugs. 47: 25-50.

10. Barredo J. C., Synold T. W., Laver, J., Relling, M. V., Pui, C. H., Priest, D. G., and Evans, W. E. (1994) Blood. 84: 564-569.

11. Barredo, J., and Moran, R. G. (1992) Mol. Pharmacol. 42: 687-694.

12. Beardsley, G. P., Moroson, B. A., Taylor, E. C., and Moran, R. G. (1989) J. Biol. Chem. 264: 328-333.

13. Bertino, J. R. (1993) J. Clin. Oncol. 11: 1-5.

14. Bollag, G., Adler, F., elMasry, N., McCabe, P., Conner, E., Thompson, P., McCormick, F., and Shannon, K. (1996) J. Biol. Chem. 271: 32491-32494.

15. Boritzki, T. J, Barlett, C. A., Zhang, C., Howland, E. F., Margosiak, S. A., Palmer, C. L., Romines, W. H., and Jackson, R. C. (1996) Invest. New Drugs 14: 295-303.

16. Brigle, K. E., Spinelia, M. J., Westin, E. H., and Goldman, I. D. (1995) Proc. Am. Assoc. Cancer Res. 36: 380.

17. Brigle, K. E., Spinella, M. J., Sierra, E. E., and Goldman, I. D. (1995) J. Biol. Chem. 270: 22974-22979.

18. Brigle, K. E., Spinella, M. J., Westin, E. H., and Goldman, I. D. (1994) Biochem. Pharmacol. 47: 337-345.

19. Carman, M. D., Schornagel, J. H., Rivest, R. S., Srimatkandada, S., Portiock, C. S., Duffy, T., and Bertino, J. R (1984) J. Clin.Oncol. 2: 16-20.

20. Chabner, B. A., Allegra, C. J., Curt, G. A., Clendeninn, N. J., Baram, J., Koizumi, S., Drake, J. C., and Jolivet, J. (1985) J. Clin. Invest. 76: 907-912.

21. Cowan, K. H., and Jolivet, J. (1984) J. Biol. Chem. 259: 10793-10800.

22. Curt, G. A., Carney, D. N., Cowan, K. H., Jolivet, J., Bailey, B. D., Drake, J. C., Dedhar, S., and Goldie, J. H. (1983) Cancer Res. 43: 4863-4871.

23. Dedhar, S., and Goldie, J. H. (1985) Biochem. Biophys. Res. Commun. 129: 536.

24. Dedhar, S., Hartley, D., and Goldie, J. H. (1985) Biochem. J. 225: 609-617.

25. Dembo, M., and Sirotnak, F. M. (1984) Acad.Press. 1:173-217.

26. Dembo, M., Sirotnak, F. M., and Moccio, D. M. (1984) J. Membrane Biol. 78: 9-1.7.

27. Dixon, K. H., Lanpher, B. C., Chiu, J., Kelley, K., and Cowan, K. H. (1994) J. Biol. Chem. 269: 17-20.

28. Drori, S., Sprecher, H., Shemer, G., Jansen, G., Goldman, I. D., and Assaraf, Y. G. (2000) Eur. J. Biochem. 267, 1-14.

29. Egan, M. G., Sirlin, S., Rumberger, B. G., Carrow, T. A., Shane, B., and Sirotnak, F. M. (1995) J. Biol. Chem. 270: 5462-5468.

30. Fabre, I., Fabre, G., and Goldman, I. D. (1984) Cancer Res. 44: 3190-3195.

31. Fan, J. Vitols, K. S., and Huennekens, F. M. (1991) J. Biol. Chem. 266: 14862-14865.

32. Fan, J. F., Pope, L. E., Vitols, K. S., and Huennekens, F. M. (1991) Biochemistry. 30:4573-4580.

33. Feinberg, A. P., and Vogelstein, B. (1983) Anal. Biochem. 132: 6-13.

34. Freemantle, S. J., Jackman, A. L., Kelland, L. R., Calvert, A. H., and Lunec, J. (1995) Br. J. Cancer. 71: 925-930.

35-. Freisheim, J. H., Ratnam, M., McAlinden, T. P., Prasad, K. M. R., Williams, F. E., Westerhof, G. R., Schornagel, J. H., and Jansen, G. (1992) Adv. Enz. Regul. 32: 17-31.

36. Freisheim, J. H., Ratnam, M., Smith, P. L., Deleamp, T. J., and Price (1988) Adv. Enz. Regul. 27:25-29.

37. Fry, D. W. and Jackson, R. C. (1986) Canc. Surveys. 5: 47-79.

38. Fry, D. W., Anderson, L. A., Borst, M., and Goldman, I. D. (1983) Cancer Res. 43: 1087-1092.

39. Fry, D. W., Yalowich, J. C., and Goldman, I. D. (1982) J. Biol. Chem. 270: 17468-17475.

40. Galivan, J. (1981(Cancer Res. 41: 1757-1762.

41. Giglia, G., Dumaz, N., Drougard, C., Avril, M. F., Daya-Grosjean, L., and Sarasin, A. (1998) Cancer Res. 58: 4402-4409.

42. Goker, E., Waltham, M., Kheradpour, A., Trippett, T., Mazumdar, M., Elisseyeff, Y., Schnieders, B., Steinherz, P., Tan, C., Berman, E., and Bertino, J. R. (1995) Blood. 86: 677-684.

43. Goldman, I. D. (1971) Ann. N. Y. Acad. Sci. 186: 400-422.

44. Goldman, I. D. (1971) Biochim. Biophys. Acta. 233: 624-633.

45. Goldman, I. D., and Matherly, L. H. (1985) Pharmacol Ther. 28: 77-102.

46. Goldman, I. D., Lichtenstein, N. S., and Oliverio, V. T. (1968) J. Biol. Chem. 243: 5007-5017.

47. Gong, M., Yess, J., Connoly, T., Ivy, S. P., Ohnuma, T., Cowan, K. H., and Moscow, J. A. (1997) Blood 89: 2494-2499.

48. Haber, D. A., and Schimke, R. T. (1981) Cell. 26:355-362.

49. Hagan, R. L., Duch, D., Smith, G. K., Hanlon, M, H., Shane, B., Freisheim, J. H., and Hynes, J. B. (1991) Biochem. Pharmacol. 41: 781-787.

50. Hanlon, M. H., and Ferone, R. (1996) Cancer Res. 56: 3301-3306.

51. Henderson, G. B., and Zevely, E. M. (1981) Biochem. Biophys. Res. Commun. 99: 163-169.

52. Henderson, G. B and Zevely, E. M. (1984) J. Biol. Chem. 259: 4558-4562.

53. Henderson, G. B. and Zevely, E. M. (1982) Biochem. Biophys. Res. Commun. 104: 474.

54. Henderson, G. B. and Zevely, E. M. (1983) Arch. Biochem. Biophys. 221: 438-446.

55. Henderson, G. B., and Zevely, E. M. (t982) Biochem. Int. 4: 493-502.

56. Henderson. G. B., Suresh, M. R., Vitols, K. S., and Huennekens, F. M. (1986) Cancer Res. 46: 1639-1643.

57. Henderson,G. B, and Strauss, B. P. (1990) Cancer Res. 50: 1709-1714.

58. Jackman, A. L., Alison, D. L., Calvert, A. H., and Harrap, K. R. (1986) Cancer Res. 46:2810-1815.

59. Jackman, A. L., and Calvert, A. H. (1995) Ann. Oncol. 6: 871-881.

60. Jackman, A. L., Kelland, L. R., Kimbell, R. I., Brown, M., Gibson, W., Aherne, G. W., Hardeastle, A., and Boyle, F. T. (1995) Br. J. Cancer. 71: 914-924.

61. Jackman, A. L., Taylor, G. A., Gibson, W., Kimbell, R., Brown, M., Calvert, A. H., Judson, I. R., and Hughes, L. R. (1991) Cancer Res. 51: 5579-5586.

62. Jackson, R. C., and Harrap, K. R. (1973) Arch. Biomed. Biophys. 158: 827-841.

63. Jackson, R. C., and Niethammer, D. (1977) Eur. J. Cancer. 13: 567-575.

64. Jaeckel, S., Epplen, J. T., Kauth, M., Miterski, B., Tschentscher, F., and Epplen, C. (1998) Electrophoresis 19: 3055-3061.

65. Jansen, G. (1988) in Antifolate Drugs: Basic Research and Clinical Practice (Jackman, A. L., ed) pp 293-321, Humana Press Inc., Totoja, N.J.

66. Jansen, G., Kathmann, I., Rademaker, B. C., Braakhuis, B. J. M., Westerhof, G. R., Rijksen, G., and Schornagel, J. H. (1989) Cancer Res. 49: 1959-1963.

67. Jansen, G., Mauritz, R., Drori, S., Sprecher, H., Kathmann, I., Bunni, M., Priest, D. G., Noordhuis, P., Schornagel, J. H., Pinedo, H. M., Peters, G. J., and Assaraf, Y. G. (1998) J. Biol. Chem. 273: 30189-30198.

68. Jansen, G., Schomagel, J. H., Kathmann, I., Westerhof, G. R., Hordijk, G. J., and van der Laan, B. F. A. M. (1992) Oncol. Res. 4: 299-305.

69. Jansen, G., Westerhof, G. R., Jarmuszewski, M. J. A., Kathmann, I., Rijksen, G., and Schomagel, J. H. (1990) J. Biol. Chem. 265: 18272-17277.

70. Jansen, G., Westerhof, G. R., Kathaman, I., Rijksen, G., and Schomagel, J. H. (1991) Cancer Chemother. Pharmacol. 28: 115-117.

71. Jansen, G., Westerhof, G. R, Kathmann, I, Rademaker, B. C., Rijksen, G., and Schorna, J. H. (1989) Cancer Res. 49: 2455-2459.

72. Kessell, D., Hall T. C., Roberts, D., and Wodinsky, I. (1965) Science 150: 752.

73. Kool, M., de Hoos, M., Scheffer, G. L., Scheper, R. J., van Eijk, M. J., Juin, J. A., Baas, F., and Borst, P. (1997) Cancer Res. 57: 3527-3547.

74. Kumar, C. K., Moyer, M. P., Dudeja, P. K., and Said, H. M. (1997) J. Biol. Chem. 272: 6226-6231.

75. Lapenta, V., Sossi, V., Gosset, P., Vassettes, C., Vitali, T., Rabatel, N., and Brahe, C. (1998) Genomics 49: 1-13.

76. Lefebvre, S. E., Rollins, S. L., and Melera, P. W. (1995) Pro. Am. Assoc. Cancer Res. 36: 376.

77. Lenz, H. J., Danenberg, K., Schnieders, B., Coeker, E., Peters, G. J., Shane, B., Bertino, J. R., and Danaberg, P. V. (1994) Oncol. Res. 6: 329-335.

78. Li, W. W., Lin, J. T., Schweitzer, B. I., Tong, W. P., Niedzwiecki D., and Bertino, J. R. (1992) Cancer Res. 52: 3908-3913.

79. Li, W. W., Waltham, M., Tong, W., Schweitzer, B. I., and Bertino, J. R. (1993) Adv. Exp. Med. Biol. 338: 635-638.

80. Lin, J. T., and Bertino, J. R. (1991) Cancer Investigation 9: 159-172.

81. Lin, J. T., Tong, W. P., Trippett, T. M., Niedzwiecki, D., Tao, Y., Tan. C. Steinherz. P., Schweitzer, B. I., and Bertino, J. R. (1991) Leukemia Research. 15: 1191-1196.

82. Lowe, K. E., Osbome, C. B., Lin, B. F., Kim, J. S., Hsu, J. C., and Shane, B. (1993) J. Biol. Chem. 268: 21665-21673.

83. Luhrs, C. A., and Slomiany, B. L. (1989) J. Biol. Chem. 264: 21446-21449.

84. Lynch, M. A., Nakashima, R., Song, H., DeGroff, V. L., Wang, D., Enomoto, T., and Weghorst, C. M.(1998) Cancer Res. 58: 4227-4232.

85. Masuda, M., I'izuka, Y., Yamazaki, M., Nishigaki, R., Kato, Y., Ni'inum, K., Suzuki, H., and Sugiyama, Y.(1997) Cancer Res. 57: 3506-3510.

86. Matherly, L. H (1995) (Georgopapadakou N H, ed.), Marcel Dekker, Inc. 453-524.

87. Matherly, L. H., and Angeles, S. M. (1994) Biochem. Pharmacol. 47: 1094-1098.

88. Matherly, L. H., Angeles, S., and McGuire, J. J. (1993) Biochem. Pharmacol. 46: 2185-2195.

89. Matherly, L. H., Angeles, S. M., and Czajkowski, C. A. (1992) J. Biol. Chem. 276: 23253-23260.

90. Matherly, L. H., Barlowe, C, K., Phillips, V. M., and Goldman, I. D. (1987) J. Biol. Chem. 262: 710-717.

91. Matherly, L. H., Barlowe, C. K., and Goldman, I. D. (1986) Cancer Res. 46: 588-593.

92. Matherly, L. H., Czajkowski, C. A., and Angeles, S. M. (1991) Cancer Res. 51: 3420-3426.

93. Matherly, L. H., Wong, S. C., Angeles, S, M., Taub, J. W., and Smith, G. K. (1994) Proc. Am. Assoc. Cancer Res. 35: 307.

94. McCloskey, D. E., McGuire, J. J., Russell, C. A., Rowan, B. G., Bertino, J. R., Pizzorno, G., and Mini, E. (1991) J. Biol. Chem. 266: 6181-6187.

95. McCormick, J. I., Susten, S. S., and Freisheim, J. H. (1981) Arch. Biochem. Biophys. 212: 311-318.

96. McGuire, J. J., Heitzrnan, K. J., Haile W. H., Russell, C. A., McCloskey, D. E., and Piper, J. R. (1993) Leukemia. 7: 1996-2003.

97. McGuire, J. J., Hsieh, P., Coward, J. K., and Bertino, J. R (1980) J. Biol. Chem. 225: 5776-5788.

98. Mini, E., Moroson, B. A., Franco, C. T., and Bertino, J. R. (1985) Cancer Res. 45: 325-330.

99. Niethammer, D. and Jackson, R. C. (1975) Eur. J. Cancer. 11: 845-854.

100. O'Connor, B. M., Jackmnan, A. L., Crossley, P. H., Freemantle, S. E., Lunec, J., and Calvert, A. H. (1992) Cancer Res. 52: 1137-1143.

101. Ohnuma, T., Lo, R. J., Scanion, K. J., Kamen, B. A., Ohnoshi, T., Wolman, S. R., and Holland, J. F. (1985) Cancer Res. 45: 1815-1822.

102. Ozaki,Y., King, R. W., and Carey, P. R., (1981) Biochemistry. 20: 3219-3225.

103. Pavlovic, M., Leffert, J. J., Russello, O., Bunni, M., Beardsley, G. P., Pierst, D. G., and Pizzorno, G. (1993) Adv. Exp. Med. Biol. 338: 775-778.

104. Pizzorno, G., Chang, Y., MeGuire, J. J., and Bertino, J. R. (1989) Cancer Res. 49: 5275-5280.

105. Pizzorno, G., Mini, E., Corronnello, M., McGuire, J. J., Moroson, B. A., Cashmore A. R., Dreyer, R. H., Lin, J. T., Mazzei, T., Periti, P., and Bertino, J. R. (1988) Cancer Res. 48: 2149-2155.

106. Ponte, P., Ng, S-Y., Engel, J., Gunning, P., and Kedes, L. (1984) Nucleic Acid Res. 12: 1687-1706.

107. Poser, R. G., Sirotnak, F. M., and Chello, P. L. (1981) Cancer Res. 41: 4441-4446.

108. Prasad, P. D., Ramamoorthy, S., Leibach, F. H., and Ganapathy, V. (1995) Biochem. Biophys. Res. Commun. 206: 681-687.

109. Price, E. M., and Freisheim, J. H. (1987) Biochemistry 26: 4757-4763.

110. Price, E. M., Sams, L., Harpring, K. M., Kempton, R. J., and Freisheim, J. H. (1986) Biochem. Pharmacol. 35: 4341-4343.

111. Ratnam, M., Marquardt, H., Duhring, J. L., and Freisheim, J. H. (1989) Biochemistry 28: 8249-8254.

112 Rhee, M. S., Galivan, J., Wright, J. E., and Rosowsky, A. (1994) Mol. Pharmacol. 45: 783-791.

113. Rhee, M. S., Wang, Y., Nair, M. G., and Galivan, J. (1993) Cancer Res. 53: 2227-2230.

114. Rosowsky, A., Lazarus, H., Yuan, G. C., Beltz, W. R., Mangini, L., Abelson, H. T., Modest, E. J., and Frei III, E. (1980) Biochem. Pharmac. 29: 648-652.

115. Rots, M. G., Pieters, R., Peters, G. J., Noordhuis, P., van Zantwijk, C. H., Kaspers, G. J., Hahlen, K., Creutzig, U., Veerman, A. J., and Jansen, G (1999) Blood 93: 1677-1683.

116. Rots, M. G., Pieters, R., Kaspers, G. J., van Zantwijk, C. H., Noordhuis, P., Mauritz, R., Veerman, A. J., Jansen, G., and Peters, G. J. (1999) Blood 93: 1067-1074.

117. Roy, K., Tolner, B., Chiao, J. H., and Sirotnak, F. M. (1998) J. Biol. Chem. 273:2526-2531.

118. Rumberger, B. G., Schmid, F. A., Otter, G. M., and Sirotnak, F. M. (1990) Cancer Commun. 2:305-310.

119. Schimke, R. T. (1984) Cell 37: 705-713.

120. Schimke, R. T. (1988) J. Biol. Chem. 263: 5989-5992.

121. Schornagel, J. H. and McVie, J. G. (1983) Cancer Treat. Rev. 10: 53-75.

122. Schuetz, J. D., Matherly, L. H.,, Westin, E. H., and Goldman, I. D. (1988) J. Biol. Chem. 263: 9840-9847.

123. Schuetz, J. D., Westin, E. H., Matherly, L. H., Pincus, R., Swerdlow, P. S., and Goldman, I. D. (1989) J. Biol. Chem. 264: 16261-16267.

124. Shane, B. (1989) Vitam. Horm. 45: 263-335.

125. Shen, D. W., Fojo, A., Roninson, I. B., Chin, J. E., Soffir, R., Pastan, I., and Gottesman, M. M. (1986) Mol. Cell. Biol. 6: 4039-4044.

126. Shih, C., Chen, V. J., Gossett, L. S., Gates, S. B., MacKellar, W. C., Habeck, L. L., Schackelford, K. A., Mendelsohn, L. G., Soose, D. J., Patel, V. F., Andiss, S. L., Bewley, J. R., Rayl, E. A., Moroson, B. A., Beardsley, G. P., Kohler, W., Ratnam, M., and Schultz, R. M. (1997) Cancer Res. 57: 1116-1123.

127. Sierra, E. E., and Goldman, I. D. (1998) Biochem. Pharmacol. 55: 1505-1512.

128. Sierra, E. E., Brigle, K. E., Spinella, M. J., and Goldman, I. D. (1997) Biochem. Pharmacol. 53: 223-231.

129. Sirotnak, F. M. (1985) Cancer Res. 45: 3992-4000.

130. Sirotnak, F. M. (1987) NCI Monogr. 5:27-35.

131. Sirotnak, F. M., Moccio, D. M., and Yang, C. H. (1984) J. Biol. Chem. 259: 13139-13144.

132. Sirotnak, F. M., Moccio, D. M., Kelleher, L. E., and Goutas, L. J. (1981) Cancer Res. 41:4447-4452.

133. Sobrero, A. and Bertino, J. R. (1986) Canc. Surveys. 5: 93-107.

134. Sprecher, H., Barr, H. M., Slotky, J. I., Tzukerman, M., Eytan, G. D., and Assaraf, Y. G. (1995) J. Biol. Chem. 270: 20668-20676.

135. Synold, T. W., Relling, M. V., Boyett, J. M., Rivera, G. K., Sandlund, J. T., Mahmoud, H., Crist, W. M., Pui, C. H., and Evans, W. E. (1994) J. Clin. Invest. 94: 1996-2001.

136. Tamura, S., Nelson, H., Tamura, A., and Nelson, N. (1998) J. Biol. Chem. 270: 28712-28715.

137. Thillet, J., Absil, J., Stone, S. R., and Pictet, R. (1988) J. Biol. Chem. 263: 12500-12508.

138. Tong, Y., Xinyue, L-C., Ercikan-Abali, E. A., Capiuax, G. M., Zhao, S-H., Banerjee, D., and Bertino, J. R. (1998) J. Biol. Chem. 273: 11611-11618.

139. Trippett, T., Schlemmer, S., Elisseyeff, Y., Goker, E., Wachter, M., Steinherz, P., Tan, C., Bermen, E., Wright, J. E., Rosowsky, A., Schweitzer, B., and Bertino, J. R. (1992) Blood. 80: 1158-1162.

140. Tse, A., and Moran, R. G. (1998) J. Biol. Chem. 273: 25944-25952.

141. Tse, A., Brigle, K. E., Taylor, S. M., and Moran, R. G. (1998) J. Biol. Chem. 273: 25953-25960.

142. Van der Laan, B. F. A. M., Jansen, G., Kathmann, I., Schornagel, J. H., and Hordijk, G. J. (1991) Eur. J. Cancer. 27: 1274-1278.

143. Wang, F. S., Aschele. C., Sobrero, A., Chang, Y., and Bertino, J. R. (1993) Cancer Res. 53: 3677-3680.

144. Wang, X., Shen, F., Freisheim, J. H., Gentry, L. F., and Ratnam, M. (1992) Biochem. Pharmacol. 44: 1898-1901.

145. Westerhof, G. R., Rijnboutt, S., Schomagel, J. H., Pinedo, H. M., Peters, G. J., and Jansen, G. (1995) Cancer Res. 55: 3795-3802.

146. Westerhof, G. R., Schornagel, J. H., Kathman, I., Jackman, A. L., Rosowsky, A., Forsch, R. A., Hynes, J. B., Boyle, F. T., Peters, G. J., Pinedo, H. M., and Jansen, G. (1995) Mol. Pharmacol. 48: 459-471.

147. Whitehead, V. M., Rosenblatt, D. S., Vuchich, M. J., Shuster, J. J., Witte, A., and Beaulieu, D. (1990) Blood. 76: 44-49.

148. Whitehead, V. M., Vuchich, M. J., Lauer, S. J., Mahoney, D., Carroll, A. J., Shuster, J. J., Esseltine, D. W., Payment, C., Look, A. T., Akabutu. J., Bowen, T., Taylor, L. D., Caniitta, B., and Pullen. D. J. (1992) Blood. 80: 1316-1323.

149. Williams, F. M. R., Murray, R. C., Underhill, T. M., and Flintoff, W. F. (1994) J. Biol. Chem. 269: 5810-5816.

150. Williams, F. M. R., and Flintoff, W. F. (1995) J. Biol. Chem. 270: 2987-2992.

151. Wong, S. C., Proefke, S. A., Bhushan, A., and Mathelry, L. H. (1995) J. Biol. Chem. 270: 17468-17475.

152. Yang, C. H., and Sirotnak, F. M. (1984) J. Membrane Biol. 79: 285-292.

153. Yang, C.-H., Sirotnak, F. M., and Mines, E. S. (1988) J. Biol. Chem. 263: 9703-9709.

154. Yang-Feng, T. L., Ma, Y-Y., Liang, R., Prasad, P. D., Leibach, F. H., and Ganapathy, V. (1995) Biochem. Biophys. Res. Commun. 210: 874-879.

155. Zhao, R., Sharina, I., and Goldman, I. D. (1999) Mol. Pharmacol., in press.

156. Zhao, R., Assaraf, Y. G., and Goldman, I. D. (1998) J. Biol. Chem. 273: 7873-7879.

157. Zhao, R., Assaraf, Y. G., and Goldman, I. D. (1988) J. Biol. Chem. 273: 19065-19071.

What is claimed is:

1. A method for the assessment of the responsiveness of a cancer patient to antifolate-containing chemotherapy, the method comprising the step of searching for a mutation or mutations in a gene associated with folate metabolism or uptake in cells derived from the patient.

2. The method of claim 1, wherein said the human gene associated with folate metabolism or uptake is the reduced folate carrier (RFC) gene.

3. The method of claim 1, wherein said gene associated with folate metabolism or uptake is selected from the group consisting of the following human genes: dihydrofolate reductase (DHFR), folylpoly γ-glutamate synthetase (FPGS), folylpoly γ-glutamate hydrolase (FPGH), glycinamide ribonucleotide transformylase (GARTF), phosphoribosylamino-imidazole-carboxamide transformylase (AICARTF), thymidylate synthase (TS), Multidrug resistance 1 (MDR1), multidrug resistance protein 1 (MRP1), multidrug resistance protein 2 (MRP2) gene and multidrug resistance protein 3 (MRP3).

4. The method of claim 1, wherein said cells are tumor cells.

5. The method of claim 1, wherein said cells are non-tumor cells.

6. The method of claim 1, further comprising the step of evaluating an effect of mutation or mutations found in said gene on antifolate and reduced folate uptake or metabolism in tumor cells of the patient.

7. The method of claim 2, further comprising the step of evaluating an effect of mutation or mutations found in said reduced folate carrier (RFC) gene on antifolate and reduced folate uptake in tumor cells of the patient.

8. The method of claim 7, wherein said step of evaluating an effect of said mutation or mutations found in said reduced folate carrier (RFC) gene on said antifolate and said reduced folate uptake in said tumor cells of the patient is effected by previous knowledge regarding said mutation or mutations.

9. The method of claim 7, wherein said step of evaluating an effect of said mutation or mutations found in said reduced folate carrier (RFC) gene on said antifolate and said reduced folate uptake in said tumor cells of the patient is effected by evaluating the effect on uptake of said antifolate and said reduced folate by cells expressing a mutated reduced folate carrier protein encoded by a polynucleotide harboring said mutation or mutations.

10. The method of claim 1, wherein said step of searching for said mutation or mutations in said gene in said cells derived from the patient is effected by a single strand conformational polymorphism (SSCP) technique.

11. The method of claim 10, wherein said single strand conformational polymorphism technique is selected from the group consisting of cDNA-SSCP and genomic DNA-SSCP.

12. The method of claim 1, wherein said step of searching for said mutation or mutations in said gene in said cells derived from the patient is effected, at least in part, by a technique selected from the group consisting of nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

13. A method for the assessment of the responsiveness of tumor cells to antifolate-containing chemotherapy, the method comprising the step of searching for a mutation or mutations in the tumor cells.

14. The method of claim 13, wherein said gene associated with folate metabolism or uptake is reduced folate carrier (RFC) gene.

15. The method of claim 13, wherein said gene associated with folate metabolism or uptake is selected from the group consisting of dihydrofolate reductase (DHFR) gene, folylpoly γ-glutamate synthetase (FPGS) gene, folylpoly γ-glutamate hydrolase (FPGH) gene, glycinamide ribonucleotide transformylase (GARTF) gene, phosphoribosylamino-imidazole-carboxamide transformylase (AICARTF) gene, thymidylate synthase (TS) gene, multidrug resistance protein 1 (MDR1) gene, multidrug resistance protein 1 (MRP1) gene, multidrug resistance protein 2 (MRP2) gene and multidrug resistance protein 3 (MRP3) gene.

16. The method of claim 13, further comprising the step of evaluating an effect of mutation or mutations found in said gene on antifolate and reduced folate uptake or metabolism in tumor cells of the patient.

17. The method of claim 14, further comprising the step of evaluating an effect of mutation or mutations found in said reduced folate carrier (RFC) gene on antifolate and reduced folate uptake in said tumor cells.

18. The method of claim 16, wherein said step of evaluating an effect of said mutation or mutations found in said gene on said antifolate and said reduced folate uptake or metabolism in said tumor cells is effected by previous knowledge regarding said mutation or mutations.

19. The method of claim 17, wherein said step of evaluating an effect of said mutation or mutations found in said reduced folate carrier (RFC) gene on said antifolate and said reduced folate uptake in said tumor cells is effected by previous knowledge regarding said mutation or mutations.

20. The method of claim 17, wherein said step of evaluating an effect of said mutation or mutations found in said reduced folate carrier (RFC) gene on said antifolate and said reduced folate uptake in said tumor cells is effected by evaluating the effect on uptake of said antifolate and said reduced folate by cells expressing a mutated reduced folate carrier protein encoded by a polynucleotide harboring said mutation or mutations.

21. The method of claim 13, wherein said step of searching for said mutation or mutations in said gene in said tumor cells is effected by a single strand conformational polymorphism (SSCP) technique.

22. The method of claim 21, wherein said single strand conformational polymorphism technique is selected from the group consisting of cDNA-SSCP and genomic DNA-SSCP.

23. The method of claim 13, wherein said step of searching for said mutation or mutations in said gene in said cells derived from the patient is effected, at least in part, by a technique selected from the group consisting of nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

24. A kit for assessing a responsiveness of a cancer patient to antifolate chemotherapy, the kit comprising a holder for holding at least one container containing oligonucleotides capable of amplifying at least one fragment of a gene associated with folate metabolism or uptake.

25. The kit of claim 24, wherein said gene associated with folate metabolism or uptake is reduced folate carrier (RFC) gene.

26. The kit of claim 24, wherein said gene associated with folate metabolism or uptake is selected from the group consisting of dihydrofolate reductase (DHFR) gene, folylpolyglutamate synthetase (FPGS) gene, folylpoly γ-glutamate hydrolase (FPGH) gene, glycinamide ribonucleotide transformylase (GARTF) gene, phosphoribosylamino-imidazole-carboxamide transformylase (AICARTF) gene, thymidylate synthase (TS) gene, multidrug resistance protein 1 (MDR1) gene, multidrug resistance protein 1 (MRP1) gene, multidrug resistance protein 2 (MRP2) gene and multidrug resistance protein 3 (MRP3) gene.

27. The kit of claim 24, further comprising a container containing a DNA polymerase enzyme.

28. The kit of claim 24, further comprising a container containing a reverse transcriptase enzyme.

29. The kit of claim 24, further comprising a container containing a mixture of dNTPs.

30. The kit of claim 24, further comprising a container containing a concentrated polymerase chain reaction buffer.

31. The kit of claim 24, wherein said oligonucleotides are selected from the group of oligonucleotides identified by SEQ ID NOs:1-20.

32. The kit of claim 24, wherein at least one of said oligonucleotides includes at least one nucleotide analog.

33. The kit of claim 24, wherein at least one of said oligonucleotides includes a labeling moiety.

34. The kit of claim 24, further comprising at least one precast gel for executing single strand conformational polymorphism.

35. The kit of claim 24, further comprising at least one container containing reagents for detection of electrophoresed nucleic acids.
